SEQUENCE LISTING (2 sequences)

SEQ ID NO: 1 - DNA, 22 bp, artificial sequence
  Forward primer for delta opioid receptor
ggtgaccaagatctgcgtgttc

SEQ ID NO: 2 - DNA, 20 bp, artificial sequence
  Reverse primer for delta opioid receptor
ttctccttggagcccgacag

(19) United States
(12) Patent Application Publication
(10) Pub. No.: US 2016/0051704 A1
(43) Pub. Date: Feb. 25, 2016

(54) MOLECULAR IMAGING PROBES FOR LUNG CANCER INTRAOPERATIVE GUIDANCE

(71) Applicants: H. Lee Moffitt Cancer Center and Research Institute, Inc., Tampa, FL (US); Arizona Board of Regents of Behalf of the University of Arizona, Tucson, AZ (US)

(72) Inventors: David L. Morse, Tampa, FL (US); Robert J. Gillies, Tampa, FL (US); Victor J. Hruby, Tucson, AZ (US)

(21) Appl. No.: 14/937,054

(22) Filed: Nov. 10, 2015

Related U.S. Application Data
(63) Continuation of application No. PCT/US2014/037712, filed on May 12, 2014.
(60) Provisional application No. 61/821,770, filed on May 10, 2013.

(51) Int. Cl. A61K 49/00; A61K 51/04

(52) U.S. Cl. CPC A61K 49/0052; A61K 49/0032; A61K 51/0497

(57) ABSTRACT

Molecular probes directed to the delta opioid receptor and associated methods of use as non-invasive diagnostics for lung cancer are presented. The molecular probes generally consist of a ligand (Dmt-Tic) that is conjugated to a detection moiety such as a fluorescent dye or a radionuclide by a linker molecule. Once the probe is administered, it may be detected by a molecular imaging device to locate tumors for treatment or removal. Also presented are novel markers for lung cancer including, but not limited to, CA9, CA12, CTAG2, CXorf61, DSG3, FAT2, KISS1R, GPR87, LYPD3, OPRD1, SLC7A11 and TMPRSS4. Probes may be developed that can target these cell surface markers.

### CROSS REFERENCE TO RELATED APPLICATIONS

This application is a continuation of International Application No. PCT/US2014/037712 with an international filing date of May 12, 2014, which claims priority to U.S. Provisional Application No. 61/821,770 filed May 10, 2013, the contents of which are hereby incorporated by reference into this disclosure.

### STATEMENT OF GOVERNMENT SUPPORT

This invention was made with Government support under Grant Nos. CA123547, CA119997 and CA097360 awarded by the National Institutes of Health (NIH). The Government has certain rights in the invention.

### FIELD OF INVENTION

This invention relates to molecular probes. Specifically, the invention describes novel molecular probes directed to novel biomarkers that can be used to target lung cancer cells.

### BACKGROUND OF THE INVENTION

Molecular imaging is a rapidly growing field whose utility has been demonstrated in numerous applications and thus its importance has been increasingly recognized. In oncology, molecular imaging is used routinely in both research and clinical settings. (James M L, Gambhir S S. A Molecular Imaging Primer: Modalities, Imaging Agents, and Applications. Physiol Rev. 2012; 92(2):897-965; Massoud T F, Gambhir S S. Molecular imaging in living subjects: seeing fundamental biological processes in a new light. Genes & Dev. 2003; 17(5):545-80; Hoffman J M, Gambhir S S. Molecular Imaging: The Vision and Opportunity for Radiology in the Future. Radiology. 2007; 244(1):39-47; Weissleder R, Pittet M J. Imaging in the era of molecular oncology. Nature. 2008; 452(7187):580-9) In the research setting, a variety of molecular imaging modalities are used with the chosen technique depending on the specific application since each one has its own advantages and limitations. In the clinic, molecular imaging with ¹⁸F-fluorodeoxyglucose (FDG) for positron emission tomography (PET) has been widely used. More recently, a number of other modalities such as optical imaging (fluorescence), magnetic resonance imaging (MRI), computed tomography (CT) and ultrasound (US) have been applied to clinical molecular imaging and new reagents for these applications are continuously being developed. (James M L, Gambhir S S. A Molecular Imaging Primer: Modalities, Imaging Agents, and Applications. Physiol Rev. 2012; 92(2):897-965)

Fluorescence imaging has been found effective for several clinical applications including intraoperative guidance. (Van Dam G M, Themelis G, Crane L M A, Harlaar N J, Pleijhuis R G, Kelder W, Sarantopoulos A, de Jong J S, Arts H J G, van der Zee A G J, Bart J, Low P S, Ntziachristos V. Intraoperative tumor-specific fluorescence imaging in ovarian cancer by folate receptor-α targeting: first in human results. Nat Med. 2011; 17(10):1315-9; Stummer W, Pichlmeier U, Meinel T, Wiestler O D, Zanella F, Reulen H J, Group A-GS. Fluorescence-guided surgery with 5-aminolevulinic acid for resection of malignant glioma: a randomised controlled multicentre phase III trial. Lancet Oncol. 2006; 7(5):392-401; Ntziachristos V, Yoo J S, Van Dam G M. Current concepts and future perspectives on surgical optical imaging in cancer. J Biomed Opt. 2010; 15(6):066024; Keereweer S, Kerrebijn J D F, van Driel P B A A, Xie B, Kaijzel E L, Snoeks T J A, Que I, Hutteman M, Van der Vorst J R, Mieog J S D, Vahrmeijer A L, Van de Velde C J H, Baatenburg de Jong R J, Lowik C W G M. Optical Image-guided Surgery—Where Do We Stand? Mol Imaging Biol. 2011; 13:199-207; Gibbs S L. Near infrared fluorescence for image-guided surgery. Quant Imaging Med Surg. 2012; 2(3):177-87) Fluorescence has the advantages of high sensitivity, ease of use, cost-effectiveness, and a lack of ionizing radiation. (James M L, Gambhir S S. A Molecular Imaging Primer: Modalities, Imaging Agents, and Applications. Physiol Rev. 2012; 92(2):897-965; Massoud T F, Gambhir S S. Molecular imaging in living subjects: seeing fundamental biological processes in a new light. Genes & Dev. 2003; 17(5):545-80)

The goal of fluorescence imaging in oncology is to differentiate the cancer tissue from the surrounding normal tissue. The two main methods for achieving this selectivity are through activatable or targeted fluorescent agents. (Hoffman J M, Gambhir S S. Molecular Imaging: The Vision and Opportunity for Radiology in the Future. Radiology. 2007; 244(1):39-47; Weissleder R, Pittet M J. Imaging in the era of molecular oncology. Nature. 2008; 452(7187):580-9)

Thus, an important aspect in the development of novel imaging agents is the selection of markers, such as enzymes for activatable agents or receptors for targeted agents, which can differentiate neoplastic tissue from normal tissue. (Nguyen Q T, Olson E S, Aguilera T A, Jiang T, Scadeng M, Ellies L G, Tsien R Y. Surgery with molecular fluorescence imaging using activatable cell-penetrating peptides decreases residual cancer and improves survival. Proc Natl Acad Sci USA. 2010; 107(9):4317-22; Urano Y, Sakabe M, Kosaka N, Ogawa M, Mitsunaga M, Asanuma D, Kamiya M, Young M R, Nagano T, Choyke P L, Kobayashi T. Rapid Cancer Detection by Topically Spraying a γ-Glutamyltranspeptidase-Activated Fluorescent Probe. Sci Transl Med. 2011; 3(110):110ra9; Mitsunaga M, Kosaka N, Choyke P L, Young M R, Dextras C R, Saud S M, Colburn N H, Sakabe M, Nagano T, Asanuma D, Urano Y, Kobayashi T. Fluorescence endoscopic detection of murine colitis-associated colon cancer by topically applied enzymatically rapid-activatable probe. Gut. 2013; 62(8):1179-86; Mieog J S D, Hutteman M, van der Vorst J R, Kuppen P J K, Que I, Dijkstra J, Kaijzel E L, Prins F, Lowik C W G M, Smit V T H B M, van de Velde C J H, Vahrmeijer A L. Image-guided tumor resection using real-time near-infrared fluorescence in a syngeneic rat model of primary breast cancer. Breast Cancer Res Treat. 2011; 128(3):679-89; Sheth R A, Upadhyay R, Stangenberg L, Sheth R, Weissleder R, Mahmood U. Improved Detection of Ovarian Cancer Metastases by Intraoperative Quantitative Fluorescence Protease Imaging in a Pre-Clinical Model. Gynecol Oncol. 2009; 112(3):616-22; Heath C H, Deep N L, Sweeny L, Zinn K R, Rosenthal E L. Use of Panitumumab-IRDye800 to Image Microscopic Head and Neck Cancer in an Orthotopic Surgical Model. Ann Surg Oncol. 2012; 19(12):3879-87; Terwisscha van Sheltinga A G T, Van Dam G M, Nagengast W B, Ntziachristos V, Hollema H, Herek J L, Schroder C P, Kosterink J G W, Lub-de Hoog M N, de Vries E G E. Intraoperative near-infrared fluorescence tumor imaging with vascular endothelial growth factor and human epidermal growth factor receptor 2 targeting antibodies. J Nucl Med. 2011; 52(11):1778-85; Themelis G, Harlaar N J, Kelder W, Bart J, Sarantopoulos A, Van Dam G M, Ntziachristos V. Enhancing Surgical Vision by Using Real-Time Imaging of αᵥβ₃-Integrin Targeted Near-Infrared Fluorescent Agent. Ann Surg Oncol. 2011; 18(12):3506-13)

Lung cancer is the second leading cause of cancer and the leading cause of cancer deaths in men and women in the United States. (Siegel R, Naishadham D, Jemal A. Cancer Statistics, 2013. CA Cancer J Clin. 2013; 63(1):11-30) The five year survival rate for this cancer is low. Following resection of lung tumors in cases that require lung conserving surgery, the course of treatment is greatly influenced by achievement of an R0 (no residual microscopic disease) relative to an R1 margin (unresected microscopic disease). There is a need for improved methods for diagnosing and treating this disease.

Fluorescently labeled targeted agents can be developed for real-time surgical guidance through the use of endoscopic instruments with fluorescence capability. In cases where lung-conservation is imperative, real-time fluorescence imaging using tumor-specific molecular imaging agents could enable the detection and removal of residual disease during surgery and could profoundly affect the course of treatment by reducing the number of patients with incomplete resections. These targeted fluorescent agents can also be used for early detection of malignant lesions by fluorescence bronchoscopy.

The delta opioid receptor (δOR) is a member of the G-protein coupled receptor family that is involved in various normal physiological processes. (Satoh M, Minami M. Molecular pharmacology of the opioid receptors. Pharmacol Ther. 1995; 68:343-64; Satoh M, Minami M. Molecular biology of the opioid receptors: structures, functions and distributions. Neurosci Res. 1995; 23(2):121-45) It is also reported to play a role in several diseases including cancer. (Zagon I S, McLaughlin P J, Goodman S R, Rhodes R E. Opioid receptors and endogenous opioids in diverse human and animal cancers. Journal of the National Cancer Institute. 1987; 79(5):1059-65; Fichna J, Janecka A. Opioid peptides in cancer. Cancer Metastasis Rev. 2004; 23(3-4):351-66; Debruyne D, Oliviera M J, Bracke M, Mareel M, Leroy A. Colon cancer cells: pro-invasive signalling. Int J Biochem Cell Biol. 2006; 38(8):1231-6) The δOR is reported to be overexpressed in lung cancer and not expressed in normal lung. (Schreiber G, Campa M J, Prabhakar S, O'Briant K, Bepler G, Patz E F. Molecular Characterization of the Human Delta Opioid Receptor in Lung Cancer. Anticancer Res. 1998; 18(3A):1787-92) Its expression has been demonstrated in human lung cancer cell lines using ligand binding assays. (Campa M J, Schreiber G, Bepler G, Bishop M J, McNutt R W, Chang K-J, Patz E F. Characterization of δ Opioid Receptors in Lung Cancer Using a Novel Nonpeptidic Ligand. Cancer Res. 1996; 56(7):1695-701; Maneckjee R, Minna J D. Opioid and nicotine receptors affect growth regulation of human lung cancer cell lines. Proc Natl Acad Sci USA. 1990; 87(9):3294-8) In addition, there are several previous studies describing the use of PET and single-photon emission computed tomography (SPECT) agents based on small molecule δOR antagonists for imaging of lung cancer. (Madar I, Bencherif B, Lever J, Heitmiller R F, Yang S C, Brock M, Brahmer J, Ravert H, Dannals R, Frost J J. Imaging δ- and μ-Opioid Receptors by PET in Lung Carcinoma Patients. J Nucl Med. 2007; 48:207-13; Collier T L, Schiller P W, Waterhouse R N. Radiosynthesis and in vivo evaluation of the pseudopeptide δ-opioid antagonist [¹²⁵I]-ITIPP(ψ). Nucl Med Biol. 2001; 28:375-81)

What is needed is a way of detecting lung cancer tumors to aid in the removal of residual cancer during surgery thus reducing the incidence of numerous resections and subsequent radiation treatments.

### SUMMARY OF INVENTION

One of the major goals of molecular imaging of cancer is to develop imaging probes that target the tumor with high specificity and selectivity. The inventors have identified a minimum set of cell-surface markers that cover 100% of lung cancers and developed fluorescent imaging probes targeted to these markers for intraoperative guidance. These probes improve the removal of residual disease during surgery and thus reduce the need for re-resection and subsequent treatment with radiation.

The inventors evaluated a synthetic peptide δ-opioid receptor (δOR) antagonist (Dmt-Tic) conjugated to two fluorescent dyes, Cy5 and IR800CW.

The inventors have synthesized a δOR-targeted fluorescent imaging agent based on a synthetic peptide antagonist (Dmt-Tic) conjugated to a Cy5 fluorescent dye (Dmt-Tic-Lys-Cy5). This agent was evaluated using a colorectal cancer cell line (HCT-116) engineered to express the δOR. It was shown to have high δOR binding affinity in vitro, demonstrated selectivity for the δOR in vitro and in vivo, and exhibited good pharmacokinetic and biodistribution profiles in vivo. It was found that Dmt-Tic demonstrates potential as a δOR-targeting ligand for the imaging of lung cancer.

To improve its potential for in vivo imaging and clinical translation, the inventors also conjugated Dmt-Tic to a near-infrared fluorescent dye (Licor IR800CW) with longer excitation and emission wavelengths than the Cy5 dye. In vivo fluorescence imaging with this agent has decreased background signal from autofluorescence and less absorption and scattering of the excitation and emission light. Binding affinity of Dmt-Tic-IR800 for the δOR was studied using lanthanide time-resolved fluorescence (LTRF) competitive binding assays in cells engineered to overexpress the δOR. In addition, lung cancer cell lines were identified with high- and non-endogenous expression of the δOR. The selectivity of Dmt-Tic-IR800 for imaging of the δOR in vivo was shown using both engineered cell lines and endogenously expressing lung cancer cells in subcutaneous xenograft models in mice. The inventors found that the δOR-specific fluorescent probe displays excellent potential for imaging of lung cancer.

Instead of a fluorescent dye, the probes may have radionuclides conjugated to the ligand to form radionuclide probes which may be used for non-invasive diagnostic imaging such using PET or CT scans.

Fluorescent Dmt-Tic probes have high selectivity and affinity for the δOR. In addition, δOR expression was verified in lung cancer which enables the use of these probes for guidance during lung cancer resection. The probes can be used for image guided surgery using fluorescence/photo or acoustic/nuclear imaging. In addition, the newly identified markers are useful for the development of new probes for lung cancer intraoperative guidance.

In an embodiment, a molecular probe having affinity for the delta opioid receptor is presented. The probe is generally comprised of a ligand, such as a synthetic peptide δ-opioid receptor (δOR) antagonist, and a detection moiety, such as a fluorescent dye or a radionuclide, conjugated to the ligand. The molecular probe may also be comprised of a linker molecule that is conjugated to both the ligand and the detection moiety to attach the detection moiety to the ligand. The linker molecule may be 3-mercaptopropionyl (Mpr), 8-amino-3,6-dioxaoctanyl (Ado), proline-lysine chains, polyethylene glycol oligomers or combinations thereof. The synthetic peptide δ-opioid receptor (δOR) antagonist may be Dmt-Tic. In an embodiment where a fluorescent dye is used, the fluorescent dye may be Cy5 or IR800CW.

A method of detecting lung cancer cells for treatment or removal is also presented comprising: providing a molecular probe directed to a specific marker expressed on the lung cancer cells wherein the markers are selected from the group consisting of CA9, CA12, CTAG2, CXorf61, DSG3, FAT2, KISS1R, GPR87, LYPD3, OPRD1, SLC7A11 and TMPRSS4; administering the molecular probe to a patient in need thereof and imaging the patient with a molecular imaging device capable of detecting a detection signal from the molecular probe wherein detection of the detection signal of the molecular probe is indicative of presence of cancer cells. The marker may be OPRD1. If the marker is OPRD1, the molecular probe may be comprised of: a synthetic peptide δ-opioid receptor (δOR) antagonist ligand such as Dmt-Tic; a linker molecule, such as 3-mercaptopropionyl (Mpr), 8-amino-3,6-dioxaoctanyl (Ado), proline-lysine chains, polyethylene glycol oligomers and combinations thereof, conjugated to the ligand; and a detection moiety, such as fluorescent dye like Cy5 and IR800CW or a radionuclide, conjugated to the linker molecule.

In a further embodiment, a method of detecting lung cancer cells in a patient is presented comprising: administering a molecular probe to the patient wherein the molecular probe specifically binds to a marker wherein the marker is δ-opioid receptor; and imaging the patient with a molecular imaging device capable of detecting a detection signal from the molecular probe wherein detection of the detection signal of the molecular probe is indicative of presence of cancer cells. The molecular probe may be comprised of: a synthetic peptide δ-opioid receptor (δOR) antagonist ligand such as Dmt-Tic; a linker molecule, such as 3-mercaptopropionyl (Mpr), 8-amino-3,6-dioxaoctanyl (Ado), proline-lysine chains, polyethylene glycol oligomers and combinations thereof, conjugated to the ligand; and a radionuclide detection moiety conjugated to the linker molecule. The radionuclide may be a positron emitting radionuclide such as gallium.

### BRIEF DESCRIPTION OF THE DRAWINGS

For a fuller understanding of the invention, reference should be made to the following detailed description, taken in connection with the accompanying drawings, in which:

FIG. 1A is a series of images depicting DNA microarray expression profile for TMPRSS4 in normal lung, lung tumors, and various other normal samples including brushed airway epithelial cells, adrenal gland, heart, kidney, liver, lymph node, and small Intestines. Data are represented as mean±sd. Asterisks indicate a significant difference between lung tumor and normal lung.

FIG. 1B is an image depicting KISS1R in normal lung, lung tumors, and various other normal samples including brushed airway epithelial cells, adrenal gland, heart, kidney, liver, lymph node, and small Intestines. Data are represented as mean±sd. Asterisks indicate a significant difference between lung tumor and normal lung.

FIG. 2A-B is a series of graphs depicting DNA microarray expression profiles for KISS1R (A) and SLC7a11 (B) in normal lung, lung tumors, and various other normal samples. Data are represented as mean±s.d. Asterisks indicate a significant difference between lung tumor and normal lung.

FIG. 2C-D is a series of graphs depicting DNA microarray expression profiles for CA12 (C) and CA9 (D) in normal lung, lung tumors, and various other normal samples. Data are represented as mean±s.d. Asterisks indicate a significant difference between lung tumor and normal lung.

FIG. 2E-F is a series of graphs depicting DNA microarray expression profiles for TMPRSS4 (E) and GPR87 (F) in normal lung, lung tumors, and various other normal samples. Data are represented as mean±s.d. Asterisks indicate a significant difference between lung tumor and normal lung.

FIG. 2G is a graph depicting DNA microarray expression profiles for LYPD3 (G) in normal lung, lung tumors, and various other normal samples. Data are represented as mean±s.d. Asterisks indicate a significant difference between lung tumor and normal lung.

FIG. 3A is a graph comparing expression in adenocarcinoma and SCC for DSG3. As shown in the figure, expression is higher in SCC than adenocarcinoma.

FIG. 3B is a graph comparing expression in adenocarcinoma and SCC for KISS1R. As shown in the figure, expression is higher in adenocarcinoma than in SCC

FIG. 3C is a graph comparing expression in adenocarcinoma and SCC for CXorf61. As shown in the figure, expression is higher in adenocarcinoma than SCC.

FIG. 4 is a table listing the expression of markers in NSCLC cell lines. mRNA expression data was analyzed for each marker.

FIG. 5A is a series of representative images of immunohistochemistry of patient samples stained for LYPD3.

FIG. 5B is a table depicting the pathology scores for the various markers.

FIG. 6 is a table summarizing the survival analyses for the lung cancer markers. Based on the Kaplan-Meier survival analyses for all 12 genes in the 444 Moffitt patients, some with multiple expression array probesets, 6 genes were observed to significantly correlate with decreased survival: CA12, CA9, CXorf61, CPR87, LYPD3 and SLC7A11. Log rank p-values from the analyses are provided demonstrating significance of the observation.

FIG. 7 is an image depicting a representative Kaplan-Meier Survival Plot for LYPD3 showing decreased survival with high expression of the LYPD3 gene. Data were analyzed using mRNA expression array data for 444 lung cancer patients compiled by the Moffitt Lung SPORE group. Data were dichotomized as having high or low expression based on the median expression value among the entire group of patients.

FIG. 8 is an image depicting the structure of Dmt-TIC.

FIG. 9A is a fluorescence image acquired 24 h post-administration of 40 nmol/kg Dmt-Tic-Cy5. The probe is selective for the δOR+ tumor. For PK experiments, mice were injected with 4.5 nmol/kg Dmt-Tic-Cy5 i.v. and images acquired at various time points from 0-168 h.

FIG. 9B is a graph depicting the mean tumor fluorescent signal obtained from the δOR− tumors and δOR+ tumors over a time course of 0-168 h post administration of 4.5 nmol/kg of Dmt-Tic-Cy5. The graph shows maximum probe uptake in the δOR+ tumor at 3 h.

FIG. 9C is a graph depicting the mean tumor fluorescent signal obtained from the δOR− tumors and δOR+ tumors over a time course of 0-168 h post administration of 4.5 nmol/kg of Dmt-Tic-Cy5. The graph shows maximum probe uptake in the δOR+ tumor at 3 h.

FIG. 9D is a graph depicting the fold of enhancement (δOR+ tumor signal/δOR− tumor signal) over time post-administration of 4.5 nmol/kg of Dmt-Tic-Cy5. The tumor signals were quantified, averaged (n≧3 for each time point), and plotted over time. The graph depicts the maximum signal fold of enhancement (27±9 s.d.) at 18 h in δOR+ relative to δOR− tumors.

FIG. 10 is a table listing immunohistochemistry of patient samples for delta opioid receptor expression.

FIG. 11A-B are representative images of immunohistochemistry of patient samples stained for the delta opioid receptor. FIG. 11A is a representative lung tumor sample that had a staining intensity of 3+. FIG. 11B is a representative normal lung sample that had a staining intensity of 2+.

FIG. 12A is a series of images depicting mice containing dorsal skin-fold window chamber tumor xenografts were injected with 45 nmol/kg Dmt-Tic-Cy5 i.v. Images were acquired continuously for 15 minutes post-injection and at various time points after. The images show uptake and extravasation of Dmt-Tic-Cy5 in the HCT-116/RFP (δOR negative) tumor in the dorsal skin-fold window chamber tumor xenograft. Dmt-Tic-Cy5 is taken up and extravasates from the rat GFP vessels within seconds post tail vein injection (left). Dmt-Tic-Cy5 is cleared by 33 minutes post tail vein injection (right). Light gray represents the RFP signal from the tumor cells, medium gray represents the GFP signal from the rat microvessels, and dark gray is the Cy5 signal from the ligand.

FIG. 12B is a series of images depicting mice containing dorsal skin-fold window chamber tumor xenografts were injected with 45 nmol/kg Dmt-Tic-Cy5 i.v. Images were acquired continuously for 15 minutes post-injection and at various time points after. The images show uptake of Dmt-Tic-Cy5 in the HCT-116/δOR tumor in the dorsal skin-fold window chamber tumor xenograft. Shown are the Cy5 signal from the ligand in dark gray (left images) and overlays of the Cy5 signal and white light channels (right images) pre-injection and 25 minutes, 45 minutes, 60 minutes, 1 hour 25 minutes, and 24 hours post-injection. The ligand was taken up by the tumor with increasing accumulation in the tumor area until 24 hours post-injection. Thus, the ligand shows specificity for the delta opioid receptor.

FIG. 13 is a graph depicting expression of the delta opioid receptor gene (OPRD1) in lung cancer cell lines. The expression of OPRD1 was quantified by qRT-PCR and normalized to the expression of β-actin. The cell lines that were positive for OPRD1 are shown in the graph. Error bars represent the standard deviation from three replicates.

FIG. 14 is a table listing a description of the cell types used.

FIG. 15 illustrates the general scheme for a general solid-phase synthetic strategy is developed to prepare fluorescent and/or lanthanide-labeled derivatives of the δ-opioid receptor (δOR) ligand H-Dmt-Tic-Lys(R)-OH. The high δ-OR affinity (Ki) 3 nM) and desirable in vivo characteristics of the Cy5 derivative 1 suggest its usefulness for structure-function studies and receptor localization and as a high-contrast noninvasive molecular marker for live imaging ex vivo or in vivo.

FIG. 16 is an image of Scheme 1.

FIG. 17 is an image of Scheme 2.

FIG. 18 is an image of Scheme 3.

FIG. 19A-B is a graph depicting (A) Competitive binding assay of Cy5 labeled ligand 1 (Ki) 3 nM; R2) 0.96). (B) Mean concentration-response data for DPDPE from MVD assay (n) 4 shown here).

FIG. 20A-C is a series of images depicting fluorescence imaging of targeted agent. Mice bilaterally implanted with HCT116 xenografts of (right) control cells, and (left) cells overexpressing δOR. (A) Mouse imaged 72 h post-intravenous (iv) injection of 100 μg of ligand 1. (B) Mouse imaged 24 h positive injection of 10 μg of ligand 1. (C) Fluorescence intensity values over whole tumor regions of interest after 72 h of clearance of 100 μg of ligand 1 and after 24 h of clearance of 10 μg of ligand 1.

FIG. 21 depicts Scheme 4 which is the synthetic route for Compound 1 (Dmt-Tic-IR800) using solid phase synthesis as described in Josan, et al. (Josan J S, Morse D L, Xu L, Trissal M, Baggett B, Davis P, Vagner J, Gillies R J, Hruby V J. Solid-Phase Synthetic Strategy and Bioevaluation of a Labeled δ-Opioid Receptor Ligand Dmt-Tic-Lys for In Vivo Imaging. Org Lett. 2009; 11(12):2479-82; Josan J S, De Silva C R, Yoo B, Lynch R M, Pagel M P, Vagner J, Hruby V J. Fluorescent and Lanthanide Labeling for Ligand Screens, Assays, and Imaging. Methods Mol Biol. 2011; 716:89-126) ⁱⁱ TFA-scavenger cocktail (TFA (91%), water (3%), triisopropylsilane (3%), and thioanisole (3%)) for 3 h; ⁱⁱⁱ 1.3 equiv IRDye® 800CW maleimide in DMSO.

FIG. 22 is a graph depicting Dmt-Tic-IR800 binds to the delta opioid receptor (δOR) in vitro. Shown is a representative curve from a representative competitive binding assay. HCT-116/δOR cells were incubated with Eu-DTPA-DPLCE (5×10⁻⁹ M) and increasing concentrations of Dmt-Tic-IR800 (1.02×10⁻¹³ to 5×10⁻⁶ M). Dmt-Tic-IR800 competes with Eu-DTPA-DPLCE for binding to the delta opioid receptor on HCT-116/δOR cells. This results in lower signals at higher concentrations of Dmt-Tic-IR800. Data are represented as mean±SEM (n=8).

FIG. 23A-B is a series of images depicting expression of the delta opioid receptor gene (OPRD1) in cell lines. A) Normalized expression of OPRD1 based on Affymetrix microarray data. OPRD1 expression was analyzed for a panel of lung cancer cell lines and shown are the values for the positive and negative cell lines used in this work. Data are represented as mean±SD (n=3 for DMS-53 and n=15 for H1299). ***, p≦0.001 B) The expression of OPRD1 was quantified by qRT-PCR and normalized to the expression of β-actin. The engineered cells are HCT-116 colorectal cancer cells that overexpress the delta opioid receptor (δOR). Also shown are the data for the parental HCT-116 cells that do not express the δOR. The endogenous cells are lung cancer cell lines that are positive (DMS-53) or negative (H1299) for OPRD1. Data are represented as mean±SD (n=3). **, p≦0.01, ****, p≦0.0001.

FIG. 24 is a series of images depicting the sequence of fluorescent in vivo images at different time points.

FIG. 25 is a graph depicting the pharmacokinetics in the engineered cells at 10 nmol/kg of Dmt-Tic-IR800.

FIG. 26A-B is a series of images depicting the in vivo imaging of the engineered cells at 10 nmol/kg of Dmt-Tic-IR800.

FIG. 27A-B is a series of images depicting Dmt-Tic-IR800 shows selectivity for the delta opioid receptor (δOR) in vivo. In vivo fluorescence imaging with Dmt-Tic-IR800 in engineered cells (A and B). A) Representative fluorescence image acquired 24 h post-administration of 10 nmol/kg Dmt-Tic-IR800. The mouse has bilateral HCT-116 (δOR) and HCT-116/δOR (δOR+) tumors in the left and right flanks, respectively. The probe is selective for the δOR+ tumor. B) The graph depicts the mean normalized fluorescence intensity obtained from the HCT-116 (δOR−) tumor and HCT-116/δOR (δOR+) tumor. Data are represented as mean±SD (n=4).

FIG. 27C-D is a series of images depicting Dmt-Tic-IR800 shows selectivity for the delta opioid receptor (δOR) in vivo. In vivo fluorescence imaging with Dmt-Tic-IR800 in endogenous lung cancer cells (C and D). C) Representative fluorescence image acquired 24 h post-administration of 40 nmol/kg Dmt-Tic-IR800. The mouse has bilateral H1299 (δOR−) and DMS-53 (δOR+) tumors in the left and right flanks, respectively. The probe is selective for the δOR+ tumor. D) The graph depicts the mean normalized fluorescence intensity obtained from the H1299 (δOR−) tumor and DMS-53 (δOR+) tumor. Data are represented as mean±SD (n=4). **, p≦0.01

FIG. 28 is a series of images depicting the sequence of fluorescent in vivo images at different time points.

FIG. 29A-B is a series of graphs depicting the pharmacokinetics in lung cancer xenografts at 40 nmol/kg of Dmt-Tic-IR800.

FIG. 30A-B is a series of graphs depicting the in vivo imaging in lung cancer cells.

FIG. 31 is a series of images depicting the ex vivo imaging of excised positive tumor, negative tumor, liver, lungs, kidneys and GI tract.

FIG. 32 is a graph depicting the biodistribution in lung cancer xenografts. The average background fluorescence signal was calculated using two mice that were not injected with Dmt-Tic-IR800CW. These values were used for background subtraction. Data are represented as mean+/−sd (n≧3 except for lungs where n=2). *p≦0.05, Positive:Negative tumor.

FIG. 33A-B are images depicting in vivo imaging of orthotopic lung tumor xenografts. (A) The mouse was injected with DMS-53 luc+ cells and tumor growth was monitored by bioluminescence imaging. The image is a representative 3D bioluminescence image acquired 17 weeks post-surgery. Fluorescence imaging of the orthotopic lung tumor xenograft was performed using a lung cancer specific imaging agent DORL-800. The mouse was injected with 40 nmol/kg DORL-800 i.v. and images were acquired in vivo on the FMT 2500 at various time points post-injection. (B) The mouse was injected with DMS-53 luc+ cells and tumor growth was monitored by bioluminescence imaging. The image is a representative fluorescence image of mouse acquired 24 h post-administration. The figure depicts the fluorescence signal obtained from a region of interest drawn around the DMS-53 luc+ (DOR+) tumor.

FIG. 34A is an image depicting the structure of DORL-800.

FIGS. 34B-C are images depicting in vivo fluorescence imaging of endogenously expressing subcutaneous lung cancer xenograft tumors following administration of DORL-800. The mice were i.v. injected with 40 nmol/kg DORL-800 and in vivo images acquired using the ART Optix MX3 at various post-injection time points. B) Representative in vivo fluorescence images of a mouse at 24 h post-administration of DORL-800. The graph (C) depicts the mean fluorescence intensity of the DMS-53 (DOR+) tumor (red), H1299 (DOR−) tumor (black), and kidneys (average of the signal from the left and right kidney) (blue) over a time course of 0-48 hours. Data are represented as mean±sd. The probe is selective for the DOR+ tumor.

FIG. 35 is an image of a SCID Hairless Outbred mouse (SHO) four weeks after DMS-53 luc+ (DOR+) cells were injected directly into the lung.

FIG. 36 is a table depicting immunohistochemistry scoring of marker expression in patient tissue samples.

FIG. 37 is an image depicting the HPLC data. As depicted in the image, the compound showed an elution time of 13.77 minutes with a linear gradient of 10-90% aqueous CH₃CN/0.1% CF₃CO₂H at a flow rate of 0.3 mL/min.

FIG. 38 is an image depicting the results of the Electrospray ionization-mass spectrometry (ESI-MS). As shown in the image, the ESI-MS in negative mode confirmed the structure of compound 1 [(M-2H)²⁻ calc. 852.7667. found 852.4524].

FIG. 39 is an image depicting Scheme 5: Standard Fmoc-Based Solid-Phase-Peptide Synthesis (SPPS) with Alloc-Lys as an Orthogonally Protected Side Chain. Steps 1, 2, and 5 are standard Fmoc-SPPS reactions. Step 3 is a standard Alloc deprotection followed by linker and partially protected DOTA coupling in Step 4. The metal chelation step is generally completed in less 2 hrs at room temperature. The metal adducts are stable to mild ESI-MS ionization energies which can be used to verify those adduct formations.

FIG. 40 is an image depicting a fluorescence image of mouse bearing an orthotopic lung cancer xenograft with endogenous levels of δOR 24 h after injection with a δOR− targeted imaging agent (Dmt-Tic-Lys-IR800).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

### DEFINITIONS

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. In some instances the term “about” refers to +/−20% of a given value. Where particular values are described in the application and claims, unless otherwise stated, the term “about” meaning within an acceptable error range for the particular value should be assumed.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a”, “an” and “the” include plural references unless the context clearly dictates otherwise.

“Patient” is used to describe an animal, preferably a mammal, more preferably a human, to whom treatment is administered, including prophylactic treatment with the compositions of the present invention. The terms “patient” and “subject” are used interchangeably herein.

The term “marker” is used herein to refer to a cell surface marker, such as an enzyme or receptor that is expressed on the surface of cells that serves as a marker of a specific cell type such as differentiating tumor cells from normal cells. Cell surface markers include, but are not limited to, enzymes for activatable agents and receptors for targeted agents. Specific examples of markers covered by the present invention include lung cancer markers including, but not limited to, CA9, CA12, CTAG2, CXorf61, DSG3, FAT2, KISS1R, GPR87, LYPD3, OPRD1, SLC7A11 and TMPRSS4. “OPRD1” is the δ-opioid receptor and such terms are used interchangeably herein.

The term “cancer”, “tumor”, “cancerous”, and malignant” as used herein, refer to the physiological condition in mammals that is typically characterized by unregulated cell growth. Examples of cancers benefited by the present invention include, but are not limited to, tumors in lung tissue.

A “molecular probe” as used herein refers to a composition that comprises a ligand that selectively binds to a specific receptor and a detection moiety conjugated to the ligand by a linker molecule. In some cases, the detection moiety is a fluorescent dye which will emit a fluorescent light when illuminated with an incident energy. In some embodiments, the receptor is the δ-opioid receptor.

The “detection moiety” as used herein may be a fluorescent dye or label, such as a chelating label, that emits a detectable signal. Alternatively, the “detection moiety” may be a radionuclide such as a positron emitting radionuclide, e.g. F-18 or Ga-68 that can be used in PET scans.

The term “ligand” as used herein refers to a molecule that binds to a receptor. Binding molecules may include peptides and polypeptides and proteins, as well as antibodies and small molecules. The ligand may be an opioid ligand in cases where the receptor is an opioid receptor, such as the dipeptide Dmt-Tic. Among the wide variety of opioid ligands known, the dipeptide Dmt-Tic represents the minimal peptide sequence that selectively interacts with δ-opioid receptor as a potent antagonist. With regard to the Dmt-Tic ligand, the N-terminus is critical for opioid receptor affinity and the free carboxylic function at the C-terminus is important for δ-opioid receptor selectivity. If another receptor is targeted, the ligand is specifically directed to have binding affinity for that particular receptor. In some instances, the term “ligand” refers to the entire compound (ligand plus detection moiety) that is bound to the receptor.

The term “peptide” as used herein refers to short polymers formed from the linking, in a defined order, of α-amino acids. The link between one amino acid residue and the next is known as an amide bond or a peptide bond. Proteins are polypeptide molecules (or consist of multiple polypeptide subunits). The distinction is that peptides are short and polypeptides/proteins are long. There are several different conventions to determine these. Peptide chains that are short enough to be made synthetically from the constituent amino acids are called peptides, rather than proteins, with one commonly understood dividing line at about 50 amino acids in length.

The term “polypeptide” as used herein refers to a compound made up of a single-chain of amino acid residues that are linked by peptide bonds. The term “protein” may be synonymous with the term “polypeptide” or may refer, in addition, to a complex of two or more polypeptides. Generally, polypeptides and proteins are formed predominantly of naturally occurring amino acids.

The term “linker” and “linker molecule” are used interchangeably herein to refer to any molecular structure that connects a detection moiety to the ligand in order to form the molecular probe. The linker may be a small linker such as 3-mercaptopropionyl (Mpr) or 8-amino-3,6-dioxaoctanyl (Ado), or a larger linker including proline-lysine chains or polyethylene glycol oligomers, or a combination of such.

The term “detectable signal” as used herein refers to a signal in an amount sufficient to yield an acceptable image using equipment that is available for clinical, laboratory, or pre-clinical use. A detectable signal may be generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “detection” of a signal as used herein refers typically to the use of a molecular imaging device such as a light detection device including, but not limited to, a charge-coupled detector that converts light energy to an electrical signal. It is known in the art that the light emissions from a source may be focused onto the detector for the formation of an image of the emitted light that may be observed visually by such as a physician. “Detection” also refers to the use of such molecular imaging devices as those described below to detect a signal from the probe according to the type of detection moiety used in the probe.

The term “molecular imaging device” as used herein refers to any device capable of detecting a signal from the molecular probe. The devices may be capable of detecting optical images such as fluorescence/light, acoustic waves, or radioisotopes. Examples of such devices include, but are not limited to, a gamma camera, PET scanner, CT scanner, SPECT scanner, MRI unit, optical imaging detector and an ultrasound machine.

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorescent molecule.

The term “dye” as used herein refers to a fluorescent molecule, i.e., one that emits electromagnetic radiation, especially of visible light, when stimulated by the absorption of incident radiation. The term includes, but is not limited to, infrared dyes such as the Licor® IR800CW dye and cyanine dyes, such as Cy5, fluorescein, indocyanine green (ICG) and derivatives thereof, pyrenes, dansyls and rhodamines (a class of dyes based on the rhodamine ring structure). Any dye that is capable of binding to the side chain, often a C-terminal residue, in a manner that has minimal influence on the ligand binding domain are contemplated by the invention.

The term “administering” as used herein refers to the process in which the probe is delivered to the patient. The probe may be systemically delivered to the target and other tissues of the host, or delivered to a localized target area of the host. Administration may be, but is not limited to, parenteral delivery including intravenous and intraarterial delivery, intraperitoneal delivery, intramuscularly, subcutaneously or by any other method known in the art.

Cell Surface Markers

The discovery of bona fide cell-surface markers for lung cancer is a key initial step in the development of lung cancer specific molecular imaging probes. Once determined, such markers may be useful targets for the development of nuclear imaging probes for the detection of metastasis, regional lymph node involvement, and to follow therapy response. Additionally, fluorescently labeled targeted probes can be developed for real-time surgical guidance through the use of endoscopic instruments with fluorescence capability. In cases where lung-conservation is imperative, real-time fluorescence imaging using tumor-specific molecular imaging probes could enable the detection and removal of residual disease during surgery and could profoundly affect the course of treatment by reducing the number of patients with incomplete resections.

Gene expression profiling was performed using DNA microarray data from patient samples of lung cancer and normal tissues. Available data sets were compiled and filtered for cell-surface, secreted, extracellular matrix, and plasma membrane genes. The resulting list of genes were ranked by their intensity and breadth of expression among the lung cancer samples relative to their differentially low expression in normal/unaffected lung samples and in other tissues of concern, e.g. liver, kidney, heart, etc. From this ranked list, twelve markers (CA9, CA12, CTAG2, CXorf61, DSG3, FAT2, KISS1R, GPR87, LYPD3, OPRD1, SLC7A11 and TMPRSS4) were selected for confirmation of protein expression by immunohistochemistry (IHC). Seven of these markers were selected based solely on their high and broad expression among the lung cancer samples relative to normal lung.

Of the markers, carbonic anhydrases (CA9 and CA12) catalyze reversible hydration of carbon dioxide. Fluorescent antibody probes and commercially available fluorescent inhibitor-based probe are available.

Cancer testis antigens (CTAG2 (LAGE-1) and CXorf61 (KK-LC-1)) show expression only in testis in normal tissue.

Cadherin family members (DSG3 and FAT2) have an important role in cellular adhesion.

G-protein coupled receptor 87 (GPR87) is a seven transmembrane protein and a member of the P2Y family.

Kiss-1 receptor (KISS1R) is also called the metastin receptor, kisspeptins receptor and G-protein coupled receptor 54. High affinity ligands as well as agonists and antagonists have been reported for KISS1R.

Ly6/PLAUR domain-containing protein 3 (LYPD3) is a glycolipid anchored membrane glycoprotein.

Solute carrier family 7 member 11 (SLC7A11) is also called the cysteine/glutamate transporter, amino acid transport system x_{c}⁻, and xCT. There is a known 18F labeled PET imaging agent.

Transmembrane protease serine 4 (TMPRSS4) participates in the regulation of cellular signaling events.

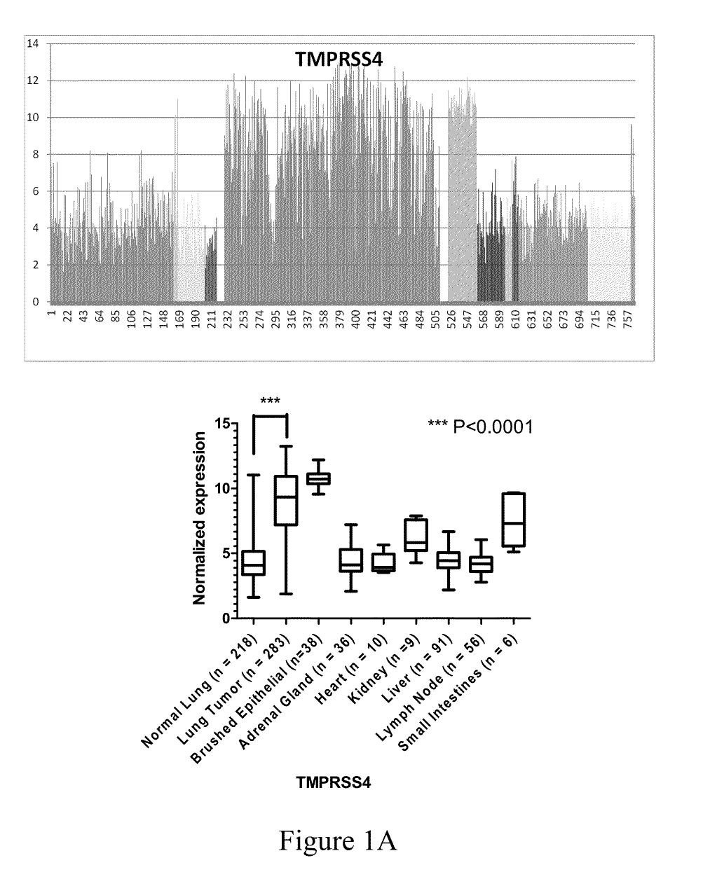

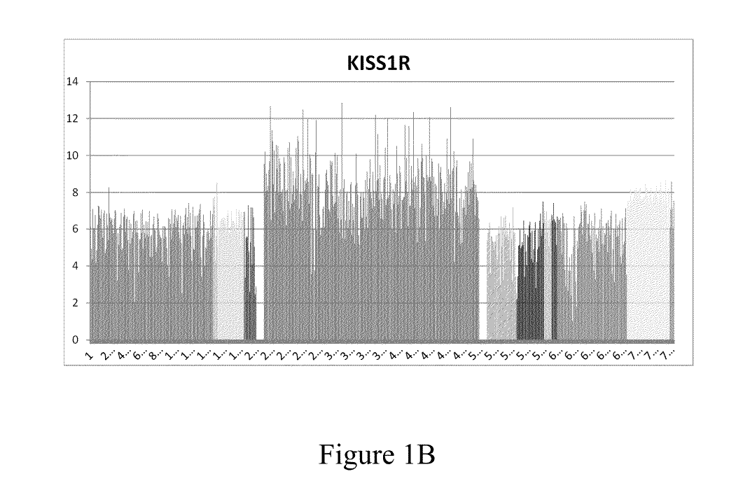

FIG. 1A shows the expression profile of the most promising of these genes, TMPRSS4. The additional five markers were selected based on their profile and that there are currently available molecular imaging probes for these markers (CA9, CA12, OPRD1 and SLC7A11), or a known high affinity ligand and structure activity relationships for the development of a probe (KISS1R). FIG. 1B shows the expression profile of KISS1R.

For the identification of new markers, gene expression profiling was performed using DNA microarray data from patient samples of lung cancer and normal tissues. Available data sets were compiled and filtered for cell-surface, secreted, extracellular matrix, and plasma membrane genes. The resulting list of genes were ranked by their intensity and breadth of expression among the lung cancer samples relative to their differentially low expression in normal/unaffected lung samples and in other tissues of concern, e.g. liver, kidney, heart, etc.

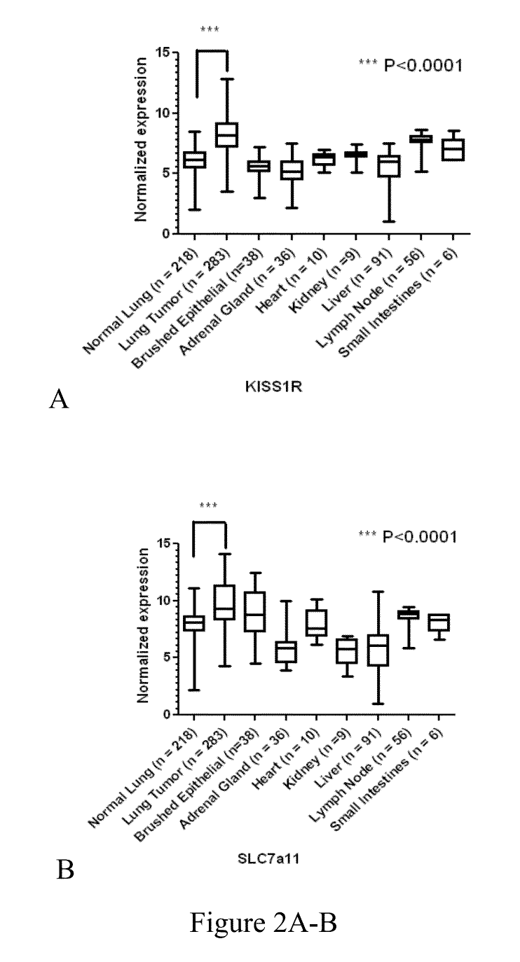

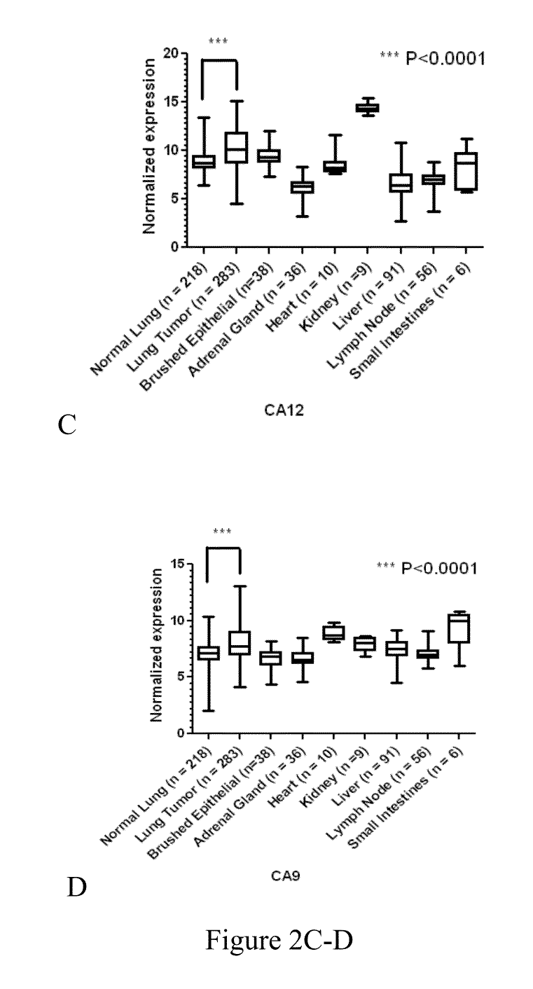

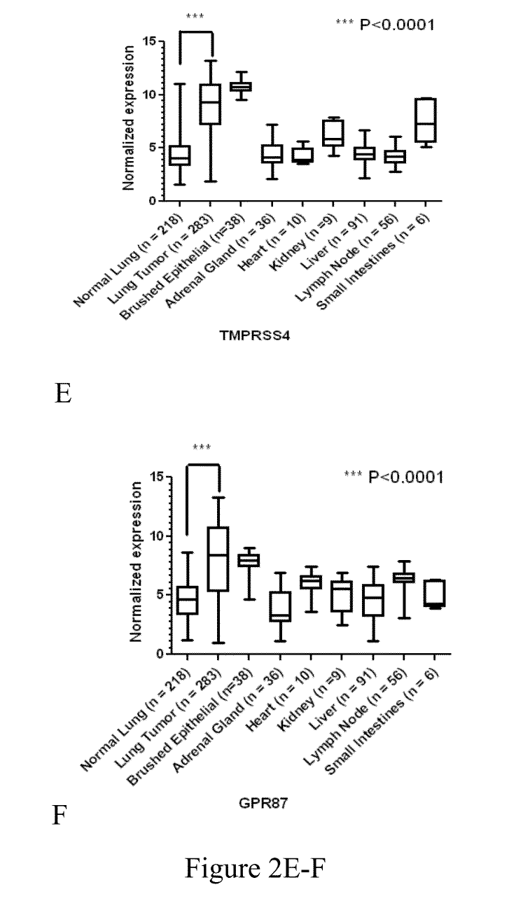

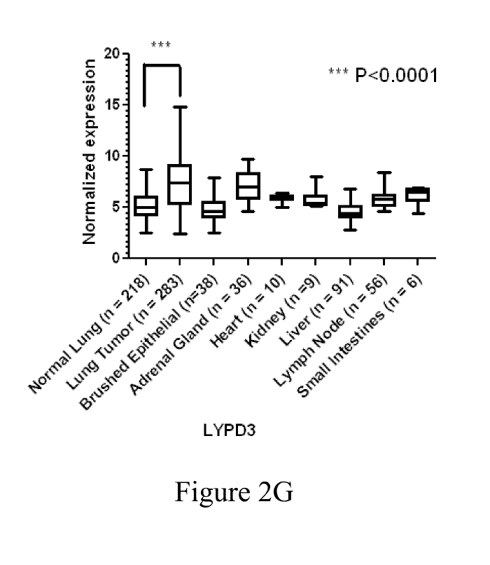

FIG. 2 is a series of graphs depicting DNA microarray expression profiles for KISS1R (A), SLC7a11 (B), CA12 (C), CA9 (D), TMPRSS4 (E), GPR87 (F), and LYPD3 (G) in normal lung, lung tumors, and various other normal samples. Data are represented as mean±s.d. Asterisks indicate a significant difference between lung tumor and normal lung.

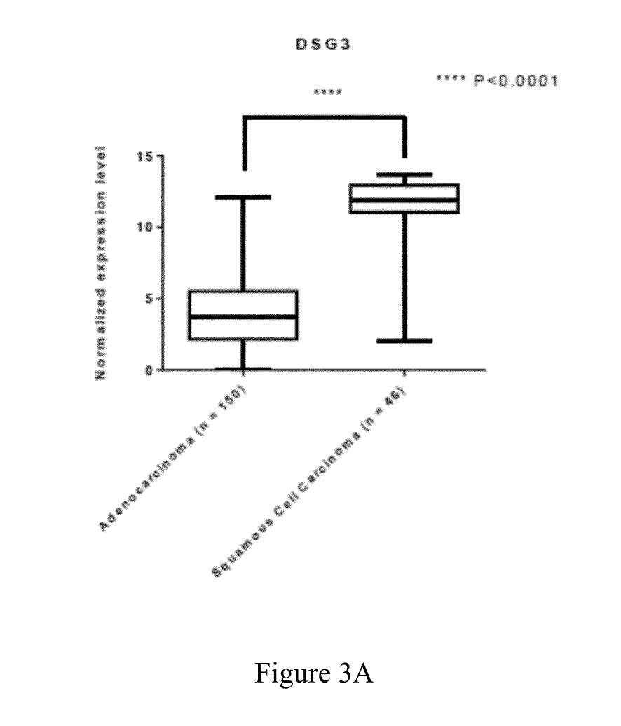

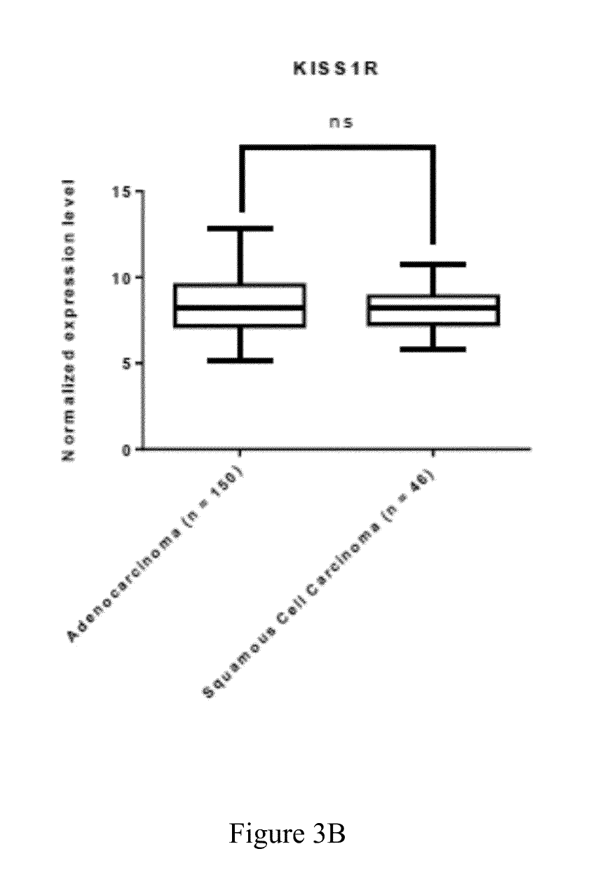

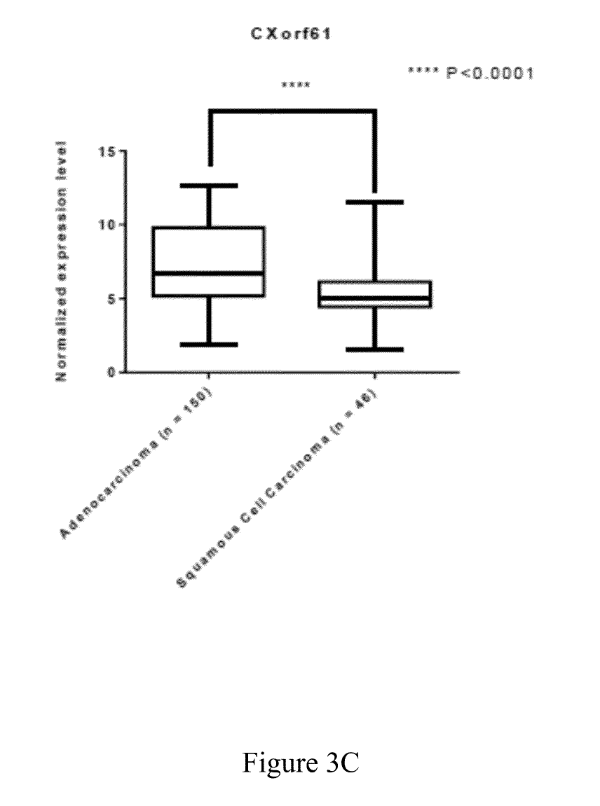

FIG. 3 is series of graphs comparing expression in adenocarcinoma and SCC. As shown in the figures, expression is higher in SCC than adenocarcinoma for all markers except KISS1R and CXorf61.

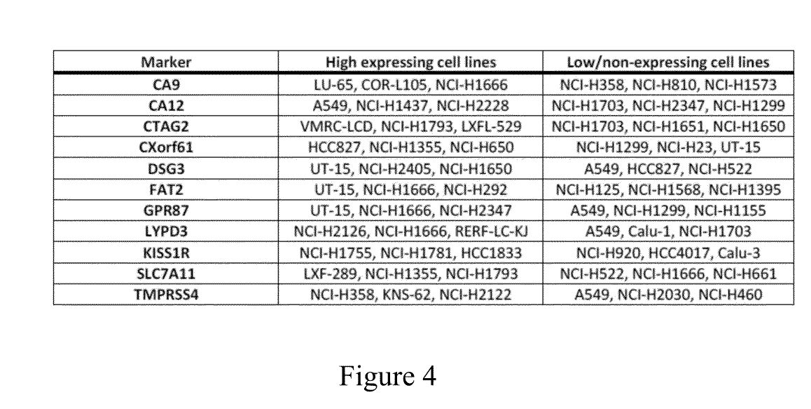

FIG. 4 is a table listing the expression of markers in NSCLC cell lines. mRNA expression data was analyzed for each marker.

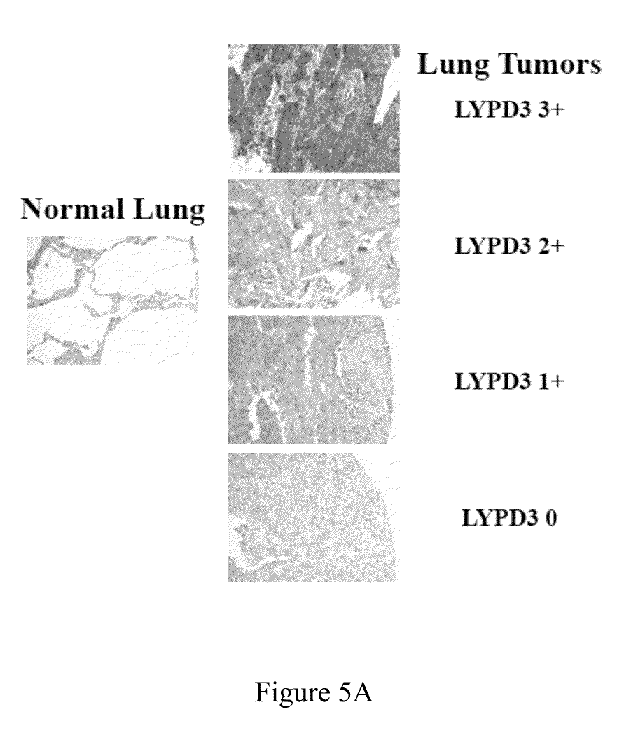

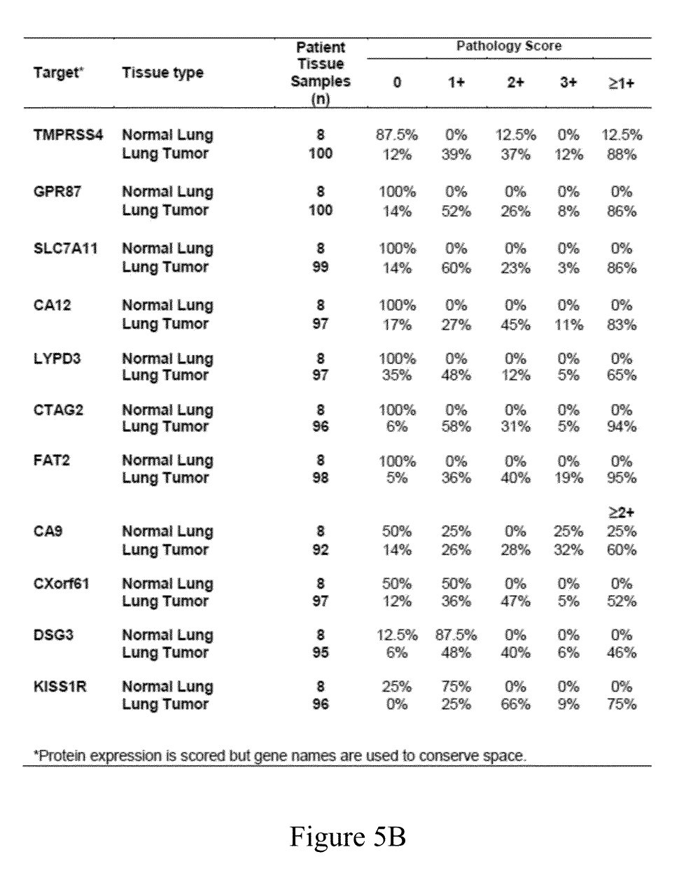

FIG. 5A is a series of representative images of immunohistochemistry of patient samples stained for LYPD3. FIG. 5B is a table depicting the pathology scores for the various markers.

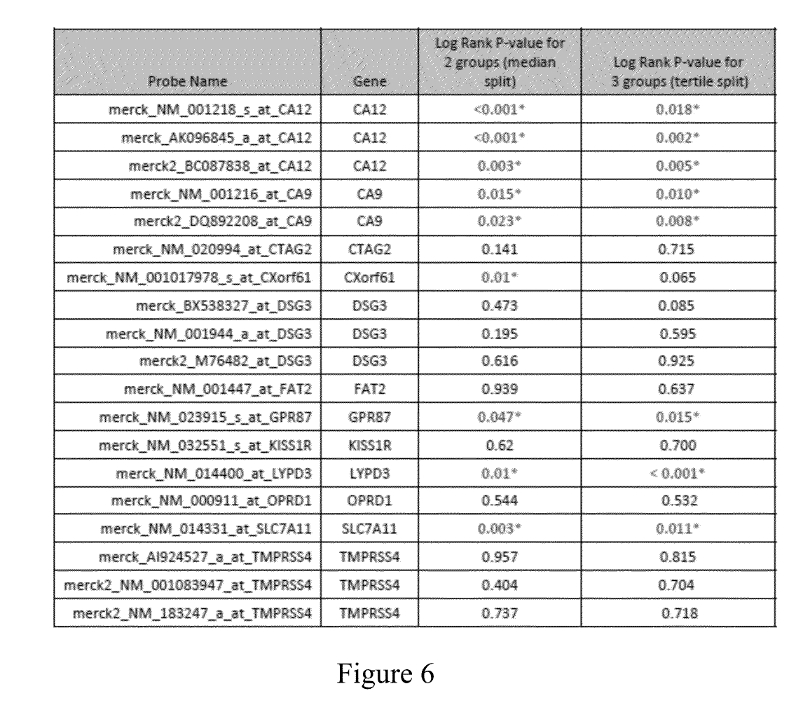

FIG. 6 is a table summarizing the survival analyses for the lung cancer markers. Based on the Kaplan-Meier survival analyses for all 12 genes in the 444 Moffitt patients, some with multiple expression array probesets, 6 genes were observed to significantly correlate with decreased survival: CA12, CA9, CXorf61, CPR87, LYPD3 and SLC7A11. Log rank p-values from the analyses are provided demonstrating significance of the observation.

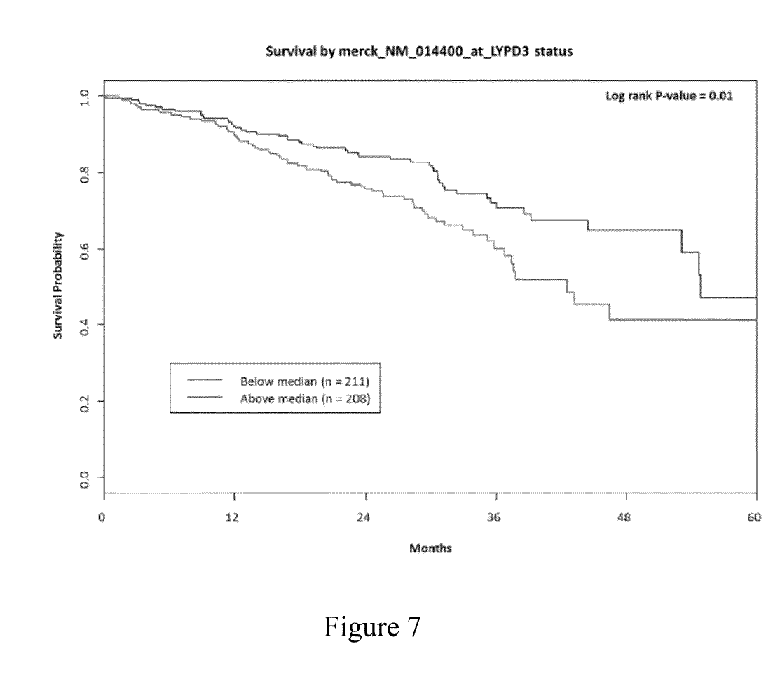

The inventors examined mRNA expression data available from 444 lung cancer patients through the Moffitt Lung SPORE group. The data was dichotomized as high and low expression of the marker based on the median cut-point and survival was compared for groups with high versus low expression of the markers. (FIG. 7) As shown in the figure, decreased survival was shown in those patients having a high expression of the LYPD3 gene. The data was also analyzed using tertiles of expression (data not shown).

The inventors have developed a high-affinity fluorescent probe specific for the delta-opioid receptor (OPRD1) based on the peptidomimetic antagonist Dmt-Tic-Lys(Cy5)-OH which has high tumor specificity and favorable pharmacokinetics and biodistribution profiles in small animal models of cancer. An ¹⁸F-glutamate derivative PET agent has also been developed that targets the x_{C}⁻ transporter (SLC7A11). High-affinity peptidomimetic ligands are known for the KiSS-1 receptor (KISS1R) that could be used to develop a novel targeted imaging probe. When protein expression is confirmed in patient samples, these newly identified markers should be useful for the development and clinical use of targeted molecular imaging probes for lung cancer.

Molecular Probes

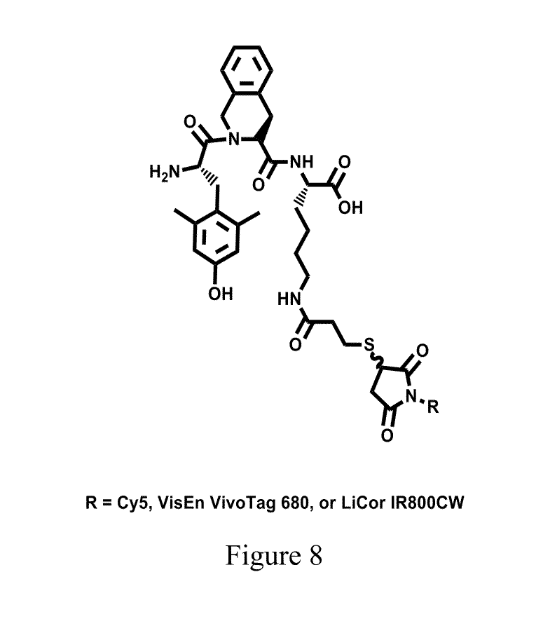

The inventors evaluated a synthetic peptide δ-opioid receptor (δOR) antagonist Dmt-Tic (FIG. 8) conjugated to two different fluorescent dyes, Cy5 and IR800CW, as further discussed below. The pharmacokinetics (PK) of Dmt-Tic-Cy5 was evaluated in a subcutaneous xenograft model using HCT-116 cells (δOR−) and HCT-116 cells that were engineered to overexpress the δOR (δOR+). Tumor uptake of Dmt-Tic-Cy5 was also studied by intravital fluorescence microscopy using a dorsal skin-fold window chamber tumor xenograft model.

Dmt-Tic-Cy5 shows high in vivo tumor selectivity with favorable PK and biodistribution profiles. The intravital imaging experiment confirmed the high binding specificity of Dmt-Tic-Cy5 and tumor cell uptake was observed by 24 h post administration. Dmt-Tic-IR800CW retains binding affinity for δOR (Kᵢ=1.43±0.24 nM, n=3). Six of the lung cancer cell lines show no expression of OPRD1 by qRT-PCR. The other five cell lines have varying levels of expression. δOR expression was found in both lung tumor samples and lung normal samples with higher expression in tumors. Seven new cell-surface markers were identified based on increased expression in lung tumors over lung normals and limited expression in other organs as discussed previously.

The binding affinity of the Dmt-Tic-IR800CW probe was evaluated in vitro using a time-resolved fluorescence competitive binding assay. The Dmt-Tic-IR800CW probe was also tested in vivo for its selectivity. A panel of 11 lung cancer cell lines was obtained and screened for expression of the delta opioid receptor gene (OPRD1) using qRT-PCR. Delta opioid receptor protein expression was verified in patient samples by immunohistochemistry of the lung cancer tissue microarray (TMA).

Dmt-Tic-Cy5

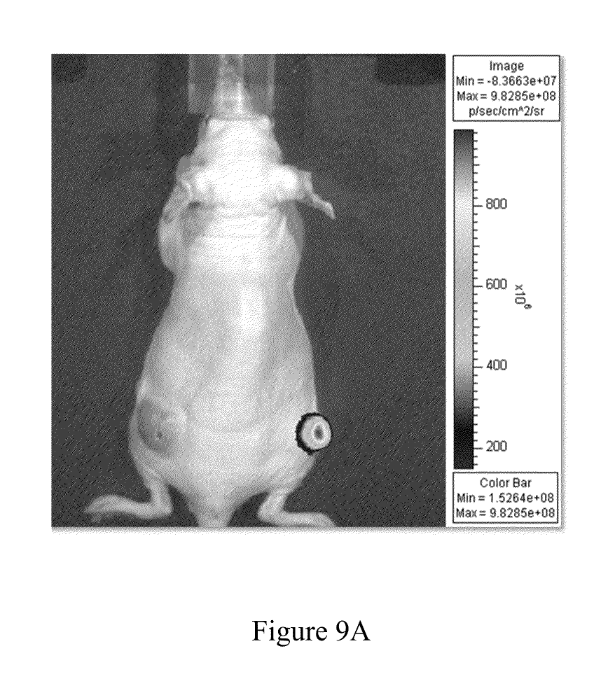

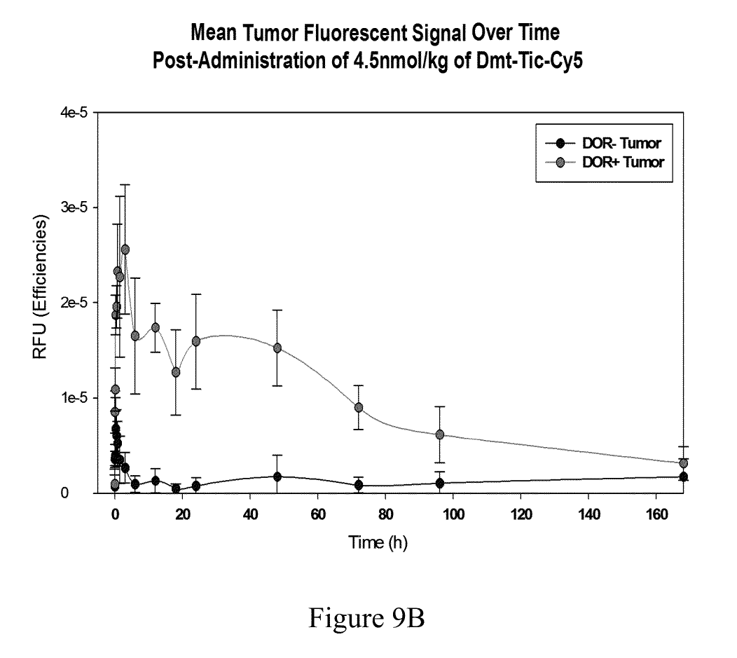

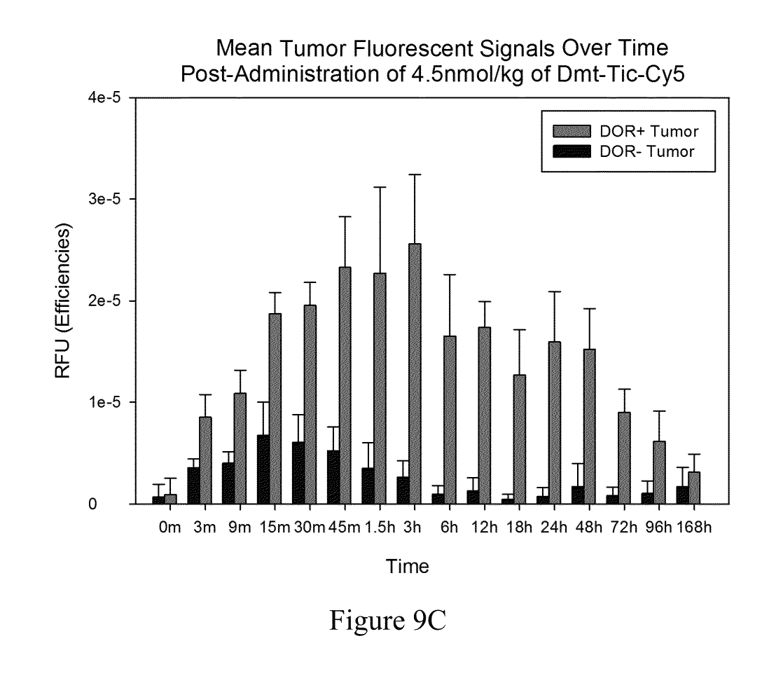

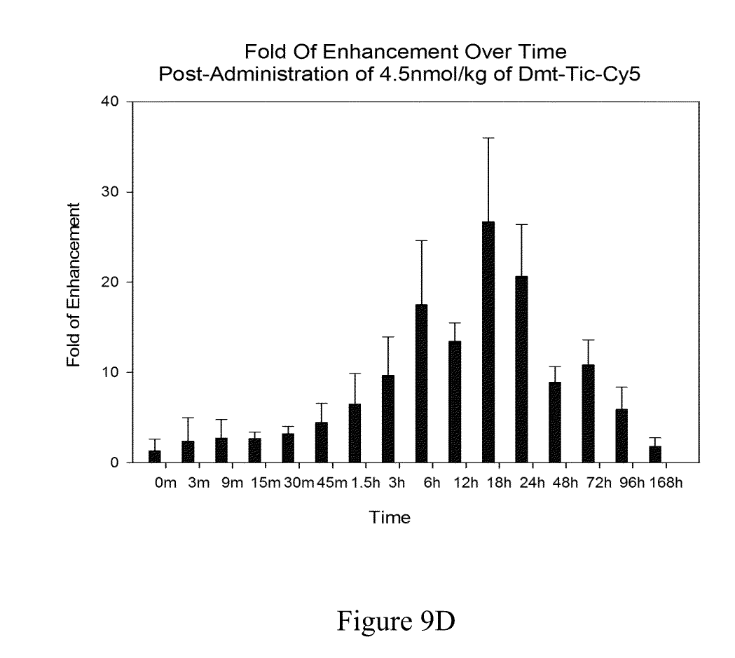

As shown in FIG. 9, fluorescence image acquired 24 h post-administration of 40 nmol/kg Dmt-Tic-Cy5. The probe is selective for the δOR+ tumor. For PK experiments, mice were injected with 4.5 nmol/kg Dmt-Tic-Cy5 i.v. and images acquired at various time points from 0-168 h (FIG. 4A). Graphs 9B and 9C depict the mean fluorescent signal obtained from the δOR− tumors and δOR+ tumors over a time course of 0-168 h. Graph 3D depicts the mean fold of enhancement (δOR+ tumor signal/δOR− tumor signal) over time. The tumor signals were quantified, averaged (n≧3 for each time point), and plotted over time. The graphs 9B and 9C show maximum probe uptake in the δOR+ tumor at 3 h. FIG. 9D depicts the maximum signal fold of enhancement (27±9 s.d.) at 18 h in δOR+ relative to δOR− tumors.

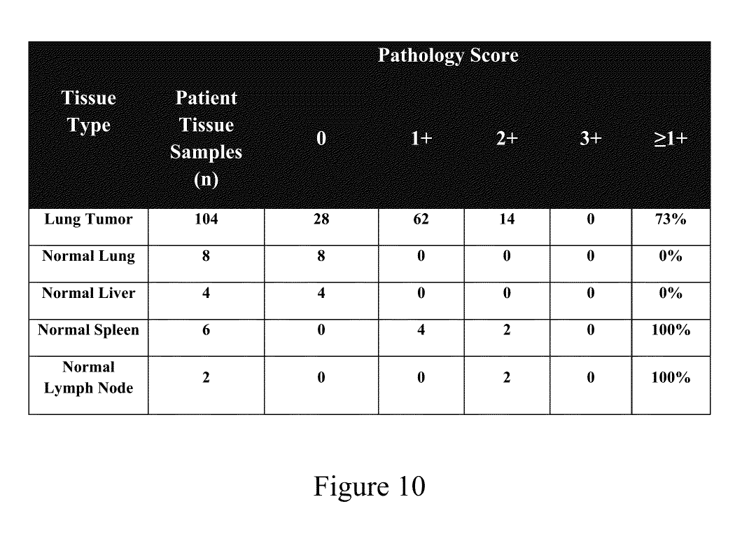

FIG. 10 is a table listing the pathology score of patient tissue samples with pathology scores ranging from 0 meaning non expression to 3+ meaning high expression.

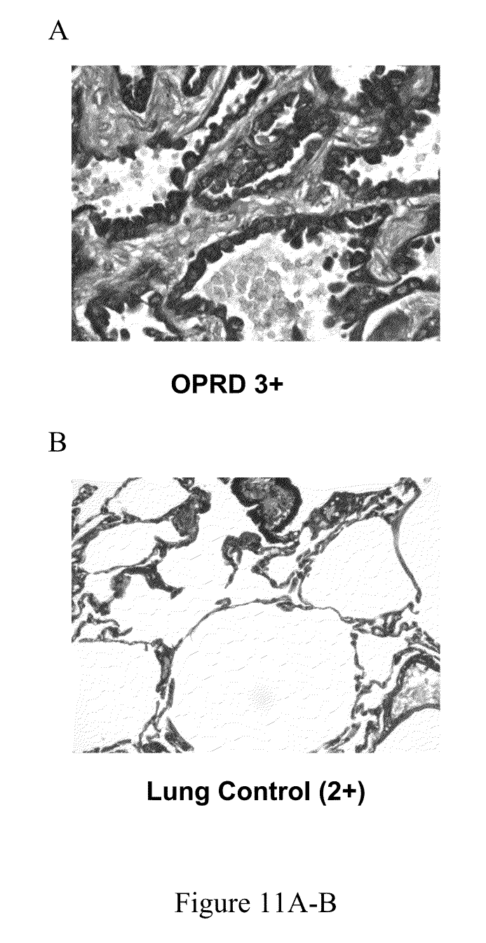

FIGS. 11A and 11B are representative images of immunohistochemistry of patient samples stained for the delta opioid receptor. FIG. 11A is a representative lung tumor sample that had a staining intensity of 3+. FIG. 11B is a representative normal lung sample that had a staining intensity of 2+.

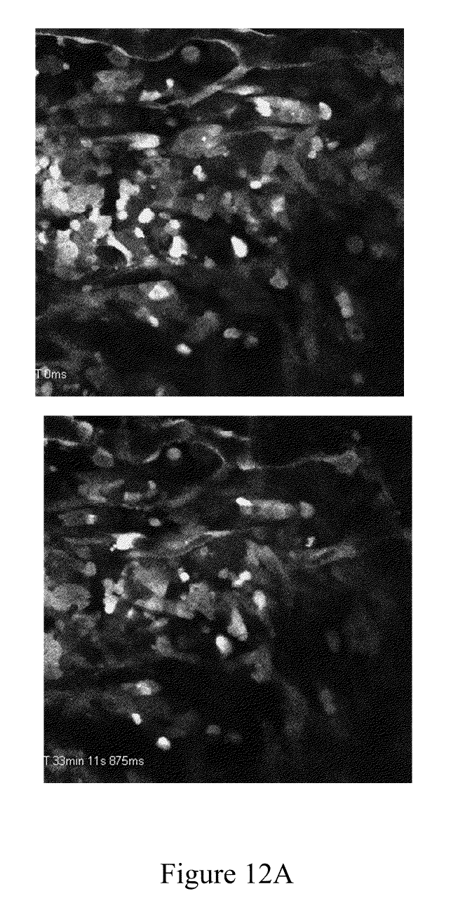

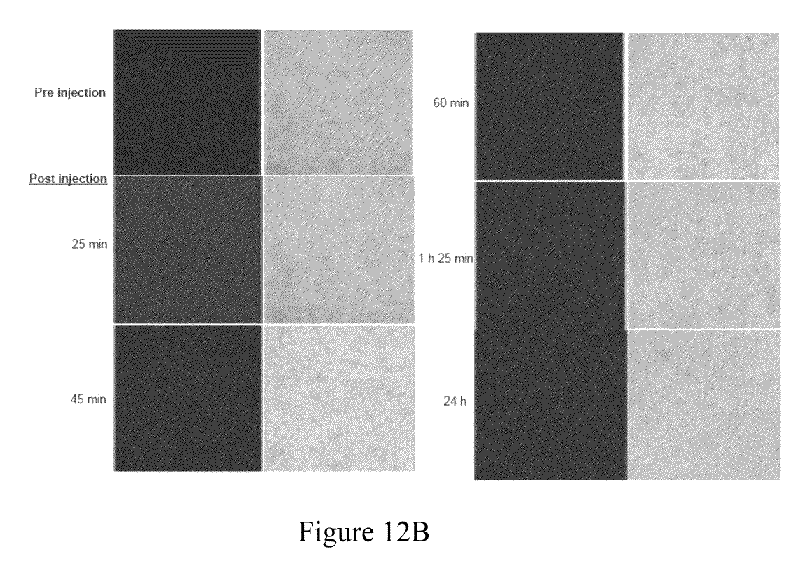

Mice containing dorsal skin-fold window chamber tumor xenografts were injected with 45 nmol/kg Dmt-Tic-Cy5 i.v. (FIG. 12) Images were acquired continuously for 15 minutes post-injection and at various time points after. FIG. 12A shows images of uptake and extravasation of Dmt-Tic-Cy5 in the HCT-116/RFP (δOR negative) tumor in the dorsal skin-fold window chamber tumor xenograft. Dmt-Tic-Cy5 is taken up and extravasates from the rat GFP vessels within seconds post tail vein injection (top). Dmt-Tic-Cy5 is cleared by 33 minutes post tail vein injection (bottom). Light gray represents the RFP signal from the tumor cells, medium gray represents the GFP signal from the rat microvessels, and dark gray is the Cy5 signal from the ligand. FIG. 12B shows images of uptake of Dmt-Tic-Cy5 in the HCT-116/δOR tumor in the dorsal skin-fold window chamber tumor xenograft. Shown are the Cy5 signal from the ligand in dark gray (left images) and overlays of the Cy5 signal and white light channels (right images) pre-injection and 25 minutes, 45 minutes, 60 minutes, 1 hour 25 minutes, and 24 hours post-injection. The ligand was taken up by the tumor with increasing accumulation in the tumor area until 24 hours post-injection. Thus, the ligand shows specificity for the delta opioid receptor.

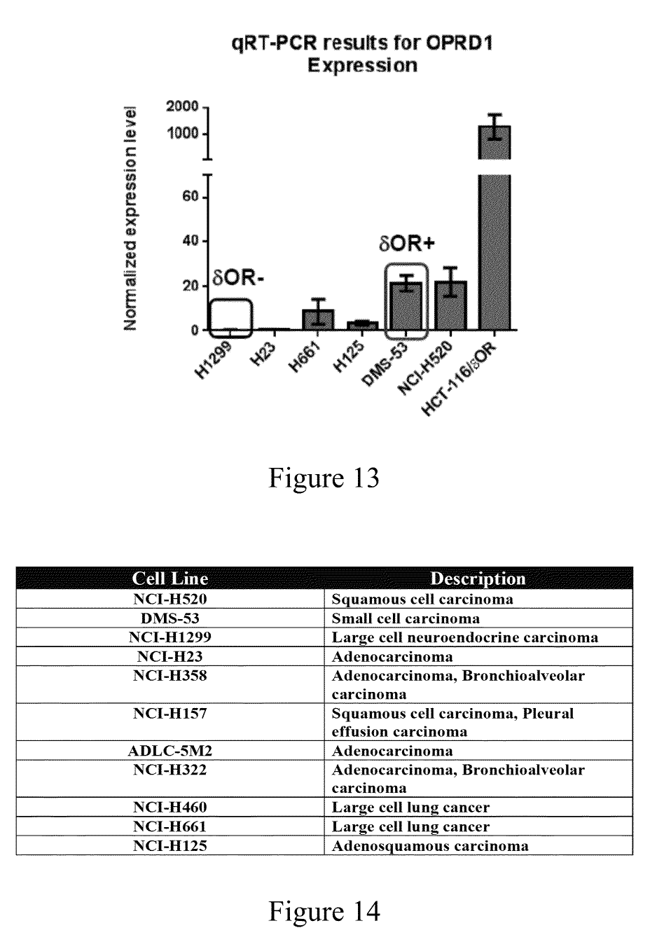

FIG. 13 shows the expression of the delta opioid receptor gene (OPRD1) in lung cancer cell lines. The expression of OPRD1 was quantified by qRT-PCR and normalized to the expression of β-actin. The cell lines that were positive for OPRD1 are shown in the graph. Error bars represent the standard deviation from three replicates.

FIG. 14 is a table listing a description of the cell types used.

The inventors have demonstrated that fluorescent Dmt-Tic probes have high selectivity and affinity for the delta opioid receptor. The increased expression of the delta opioid receptor in lung cancer has also been verified, as demonstrated for both cell lines and patient samples. This should enable the use of these probes for guidance during lung cancer resection. The newly identified markers should be useful for the development of new probes for lung cancer intraoperative guidance.

Labeling of Opioid Ligands

Labeling of opioid peptides remains an active area of research as pharmacological tools to study opioid receptor structure and function, as well as for imaging. (Lipkowski, A. W.; Misicka, A.; Kosson, D.; Kosson, P.; Lachwa-From, M.; Brodzik-Bienkowska, A.; Hruby, V. J. Life Sci. 2002, 70, 893-897; Navratilova, E.; Waite, S.; Stropova, D.; Eaton, M. C.; Alves, I. D.; Hruby, V. J.; Roeske, W. R.; Yamamura, H. I.; Varga, E. V. Mol. Pharmacol. 2007, 71, 1416-1426; Lever, J. R. Curr. Pharm. Des. 2007, 13, 33-49) Most labeled opioids for in vivo imaging have been designed to be lipophilic to permit passage across the blood-brain barrier (BBB). However, opioid receptors have also been implicated to play a role in a variety of cancers, cardiovascular diseases, and gastrointestinal disorders. (Zagon, I. S.; McLaughlin, P. J.; Goodman, S. R.; Rhodes, R. E. J. Natl. Cancer Inst. 1987, 79, 1059-1065; Schreiber, G.; Campa, M. J.; Prabhakar, S.; O'Briant, K.; Bepler, G.; Patz, E. F. Anticancer Res. 1998, 18, 1787-1792; Fichna, J.; Janecka, A. Cancer Metastasis Rev. 2004, 23, 351-366; Debruyne, D.; Oliveira, M. J.; Bracke, M.; Mareel, M.; Leroy, A. Int. J. Biochem. Cell Biol. 2006, 38, 1231-1236; Villemagne, P. S.; Dannals, R. F.; Ravert, H. T.; Frost, J. J. Eur. J. Nucl. Med. Mol. Imaging 2002, 29, 1385-1388; Pol, O.; Palacio, J. R.; Puig, M. M. J. Pharmacol. Exp. Ther. 2003, 306, 455-462)

Further, recent research promises newer paradigms of opioid analgesia acting outside the central nervous system. (Stein, C.; Schafer, M.; Machelska, H. Nat. Med. 2003, 9, 1003-1008) Therefore, there is an increasing need to develop labeled opioid ligands and establish synthetic strategies, especially solid-phase approaches, for in vivo imaging of peripherally as well as centrally restricted opioid receptors. (Ryu, E. K.; Wu, Z.; Chen, K.; Lazarus, L. H.; Marczak, E. D.; Sasaki, Y.; Ambo, A.; Salvadori, S.; Ren, C.; Zhao, H.; Balboni, G.; Chen, X. J. Med. Chem. 2008, 51, 1817-1823) These ligands could also be useful for in vitro studies such as altered opioid receptor expression profiles observed in patients with morphine dependence and in hypertrophic scars with associated nociceptive pain. (Narita, M; Funada, M.; Suzuki, T. Pharamaol. Ther. 2001, 89, 1-15; Cheng, B; Liu, H. W.; Fu, X. B.; Sheng, Z. Y.; Li, J. F. Br. J. Dermatol. 2008, 158, 713-720)

Among the wide variety of opioid ligands known, the dipeptide Dmt-Tic represents the minimal peptide sequence that selectively interacts with δ-opioid receptor as a potent antagonist (Kiδ) 0.022 nM; pA2) 8.2). (Dmt: 2′,6′-dimethyl-L-tyrosine; Tic: 1,2,3,4-tetrahydroisoquinoline-3-carboxylic acid; Salvadori, S.; Attila, M.; Balboni, G.; Bianchi, C.; Bryant, S. D.; Crescenzi, O.; Guerrini, R.; Picone, D.; Tancredi, T.; Temussi, P. A.; Lazarus, L. H. Mol. Med. 1995, 1, 678-689) Numerous derivatives of Dmt-Tic have been reported with either agonist or antagonist properties, μ- or δ-opioid selectivities, or mixed μ,δ activities, which makes it an ideal candidate for labeling. (Bryant, S. D.; Jinsmaa, Y.; Salvadori, S.; Okada, Y.; Lazarus, L. H. Biopolymers (Pept. Sci) 2003, 71, 86-102)

Opioid peptide ligands with fluorescent functionalities such as rhodamine, pyrene, dansyl, and fluorescein have been described before. (Lipkowski, A. W.; Misicka, A.; Kosson, D.; Kosson, P.; Lachwa-From, M.; Brodzik-Bienkowska, A.; Hruby, V. J. Life Sci. 2002, 70, 893-897; Navratilova, E.; Waite, S.; Stropova, D.; Eaton, M. C.; Alves, I. D.; Hruby, V. J.; Roeske, W. R.; Yamamura, H. I.; Varga, E. V. Mol. Pharmacol. 2007, 71, 1416-1426; Lever, J. R. Curr. Pharm. Des. 2007, 13, 33-49; Hazum, E.; Chang, K-J.; Shechter, Y.; Wilkinson, S.; Cuatrecasas, P. Biochem. Biophys. Res. Commun. 1979, 88, 841-846; Mihara, H.; Lee, S.; Shimohigashi, Y.; Aoyagi, H.; Kato, T.; Izumiya, N.; Costa, T. FEBS Lett. 1985, 193, 35-38; Berezowska, I.; Chung, N. N.; Lemieux, C.; Zelent, B.; Szeto, H. H.; Schiller, P. W. Peptides 2003, 24, 1195-1200; Kumar, V.; Aldrich, J. V. Org. Lett. 2003, 5, 613-616; Balboni, G.; Salvadori, S.; Piaz, A. D.; Bortolotti, F.; Argazzi, R.; Negri, L.; Lattanzi, R.; Bryant, S. D.; Jinsmaa, Y.; Lazarus, L. H. J. Med. Chem. 2004, 47, 6541-6546)

The inventors chose a cyanine (Cy) dye as it is highly fluorescent and water-soluble, as well as providing a significant advantage over other optical labels for in vivo imaging. For example, the Cy5 fluoresces in the far-red region of the visible spectrum and thus is ideal for minimizing background artifacts. Labeling of opioid peptides generally cannot involve the NR-terminus since this is critical for the opioid receptor affinity (“message region”). (Hruby, V. J.; Gehrig, C. A. Med. Res. Rev, 1989, 9, 343-401; Aldrich, J. V.; Vigil-Cruz, S. C. Burger's Medicinal Chemistry and Drug Discovery, 6th ed.; Abraham, D. J., Ed.; Wiley & Sons: New York, 2003; Vol. 6, Chapter 7, p 329) Further, a free carboxylic function at the C-terminus is important to maintain high δ-receptor selectivity. (Salvadori, S.; Attila, M.; Balboni, G.; Bianchi, C.; Bryant, S. D.; Crescenzi, O.; Guerrini, R.; Picone, D.; Tancredi, T.; Temussi, P. A.; Lazarus, L. H. Mol. Med. 1995, 1, 678-689; Balboni, G.; Onnis, V.; Congiu, C.; Zotti, M.; Sasaki, Y.; Ambo, A.; Bryant, S. D.; Jinsmaa, Y.; Lazarus, L. H.; Trapella, C.; Salvadori, S. J. Med. Chem. 2006, 49, 5610-5617; Balboni, G.; Salvadori, S.; Guerrini, R.; Negri, L.; Giannini, E.; Bryant, S. D.; Jinsmaa, Y.; Lazarus, L. H. J. Med. Chem. 2004, 47, 4066-4071; Hruby, V. J.; Gehrig, C. A. Med. Res. ReV. 1989, 9, 343-401; Aldrich, J. V.; Vigil-Cruz, S. C. Burger's Medicinal Chemistry and Drug Discovery, 6th ed.; Abraham, D. J., Ed.; Wiley & Sons: New York, 2003; Vol. 6, Chapter 7, p 329) Thus, the label must be attached on the side chain, often a C-terminal residue, in a manner that has minimal influence on the ligand binding domain. A solid-phase strategy to prepare Dmt-Tic ligands and their labeled analogues linked at the C-terminus lysine side chain via small linkers. In this context, hydrophilic components such as spacers and labels were employed for peripheral retention, lower nonspecific uptake, and faster blood clearance of the ligand. (Calculated log D of compound 1 reveals a value of 1.01 at pH 7.4 (log D: 2.6, 1.35, 1.16 at pH 1.5, 5.0, 6.5, respectively); Duval, R. A.; Allmon, R. L.; Lever, J. R. J. Med. Chem. 2007, 50, 2144-2156)

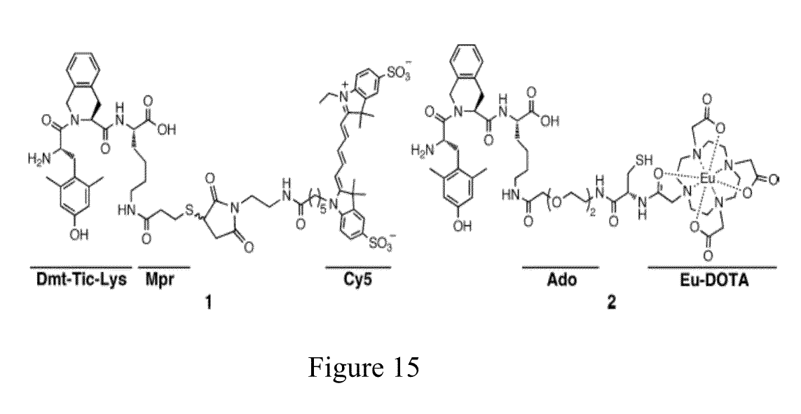

The bioevaluation of the Cy5 probe 1 as a representative example is described, and the flexibility of the synthetic scheme to prepare dual-modality agents is highlighted. FIG. 15 illustrates the general scheme for a general solid-phase synthetic strategy is developed to prepare fluorescent and/or lanthanide-labeled derivatives of the δ-opioid receptor (δOR) ligand H-Dmt-Tic-Lys(R)-OH. The high δ-OR affinity (Ki) 3 nM) and desirable in vivo characteristics of the Cy5 derivative 1 suggest its usefulness for structure-function studies and receptor localization and as a high-contrast noninvasive molecular marker for live imaging ex vivo or in vivo.

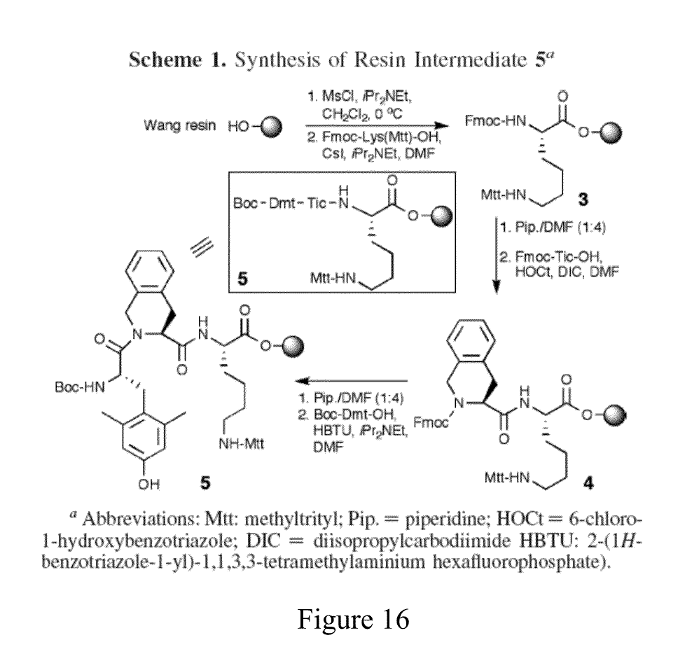

An easy, robust, and scalable synthetic route to label Dmt-Tic ligands was developed based only on commercially available reagents and a labeling scheme that could be performed on-resin or in solution as desired. Many commercially available labeling moieties contain an activated carboxylic acid (e.g., N-hydroxy succinimide (NHS) ester derivative) or a maleimide that readily react with an amine or a thiol, respectively. Therefore, the synthetic scheme was designed for labeling H-Dmt-Tic-Lys(R)-OH by a maleimide derivative of Cy5 and an NHS ester of the lanthanide chelator DOTA (1,4,7,10-tetraazacyclododecane-N,N′,N″,N′″-tetracetic acid). For optimal spacing between the ligand and the probe, small linkers such as 3-mercaptopropionyl (Mpr) and 8-amino-3,6-dioxaoctanyl (Ado) were employed. The synthesis was started with esterification of NR-Fmoc-Lys(Nε-Mtt)-OH onto Wang resin (Scheme 1 shown in FIG. 16). This was achieved from Wang resin, which was mesylated using an 8-fold excess of MsCl at 0° C. to activate OH groups, followed by coupling with Fmoc-Lys(Mtt)-OH. In a 50 mL bottle containing 1 g of Wang resin (0.93 mmol/g) with a magnetic stir bar, dry CH2Cl2 was added to swell the resin for 1 h. The solvent was removed, the bottle closed with a septum and flushed with nitrogen, and iPr2NEt (9 equiv, 1.4 mL) in 15 mL of CH2Cl2 was added. The resin slurry was cooled to 0° C. followed by dropwise addition of MsCl (8 equiv, 0.57 mL) in 2 mL of CH2Cl2. The reaction was stirred for 20 min, the ice-bath was removed, and the stirring was continued for another 20 min (rt). The resin was then transferred to a syringe reactor and washed with dry CH2Cl2 and dry DMF. Fmoc-Lys(Mtt)-OH (2 equiv, 1.2 g), CsI (2 equiv, 0.5 g), iPr2NEt (2 equiv, 0.32 mL) in ca. 10 mL of dry DMF were added, and the reaction was stirred overnight at rt.

The NR-Fmoc protection from 3 was removed with piperidine in DMF, and Fmoc-Tic-OH was coupled using standard NR-Fmoc/tBu strategy of solid-phase peptide synthesis to give intermediate 4. The resin was NR-Fmoc deprotected with piperidine/DMF (1:4) and then washed with DMF, CH2Cl2, 0.2 M HOBt/DMF, and DMF. Fmoc-Tic-OH (3 equiv), HOCt (3 equiv), and DIC (6 equiv) in DMF were then added, and the reaction was stirred for 2 h.

For final coupling, Boc-Dmt-OH was used since a free N terminal peptide can be directly obtained after final acidic cleavage. Additionally, a choice of NR-terminal Boc protection prevents against premature Fmoc deprotection by free NH2 groups released on the side chain of lysine and any consequent cyclative elimination (dioxopiperazine) of Dmt-Tic from Dmt-Tic-Lys(R)-resin. (Caspasso, S.; Sica, F.; Mazzarella, L.; Balboni, G.; Guerrini, R.; Salvadori, S. Int. J. Pep. Protein Res. 1995, 45, 567-573) Lastly, Boc is smaller than Fmoc, facilitating a faster coupling rate. Despite that, Boc-Dmt coupling to the sterically hindered Tic-Lys(R)-resin was challenging. The coupling has to be mediated via strong HBTU activation accelerated by microwave. Boc-Dmt-OH (3 equiv), HBTU (3 equiv), and iPr2NEt (6 equiv) in DMF were added to the resin, and the reaction was heated in a household microwave for 3 s. The reaction was stirred until it cooled to rt; the heating was repeated (5×), and the resin was stirred for another 2 h. Also, there is a conceptual disadvantage of using an unprotected phenolic group on Dmt. The reaction leads to Dmt self-condensation, forming small amounts of Dmt-oligomers. Nonetheless, the formed phenolic esters are susceptible to mild aminolysis and can be selectively removed by treatment with 50% piperidine in CH2Cl2:MeOH (5:1) before acidic cleavage.

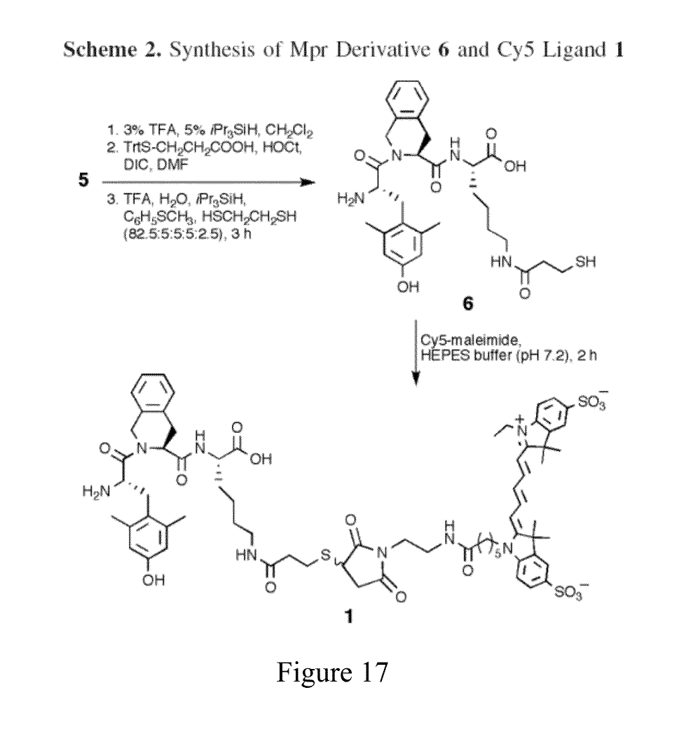

Following Dmt coupling to give intermediate 5, the coupling of dyes and chelating agents can be performed on the resin or in solution. For Cy5 labeling, more cost-effective conjugation of dye in solution was preferred via a thiolmaleimide reaction. 3-Mercaptopropionyl was chosen as a small linker for C-terminal attachment as the dye possesses a 12-atom linker with maleimide at the end. Thus, Trt-SCH2CH2COOH was coupled to 5 using HOCt/DIC protocol and then cleaved with acidic cocktail (82.5% TFA, 5% H2O, 5% iPr3SiH, 5% thioanisole, and 2.5% ethanedithiol) to give ligand 6 (Scheme 2 shown in FIG. 17). The compound was purified by preparative HPLC, and the Cy5 dye was conjugated to the peptide in solution to give ligand 1. Ligand 8 was dissolved in HEPES buffer (pH 7.2), and 1.3 equiv of Cy5-maleimide was added in aliquots until full conversion was achieved as monitored on analytical HPLC. The labeled ligand was then separated with SPE.

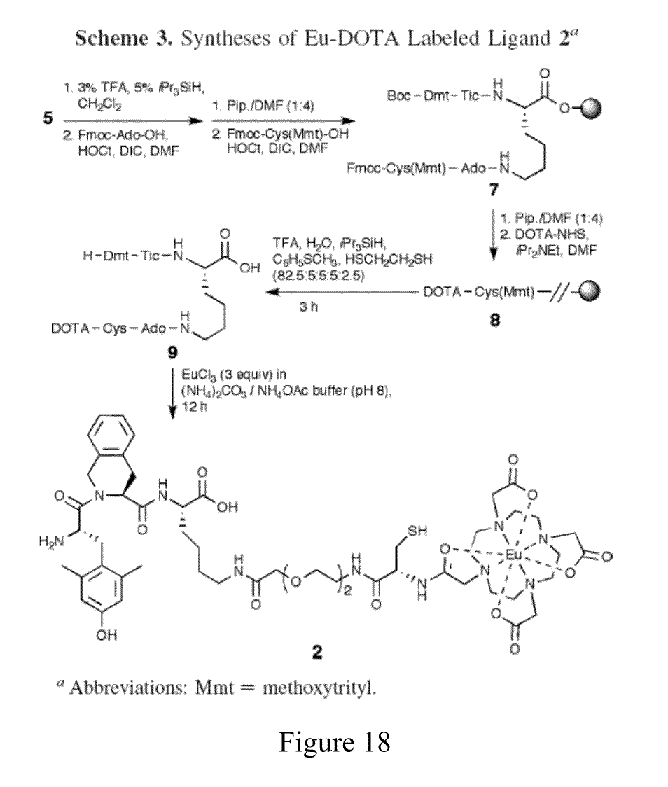

The on-resin labeling was tested by synthesizing a DOTA chelate as shown in Scheme 3 (FIG. 18). The Mtt protection on lysine was removed with 3% TFA and 5% iPr3SiH in CH2Cl2. Here the inventors employed a bifunctional handle to investigate its utility for coupling commonly available labeling moieties, for dual-modality labeling (e.g., optical/magnetic), for coupling to nanoparticles with lanthanide labels, and for preparing dimeric ligands at a later stage (unpublished data). For this purpose, the synthetic scheme was designed to incorporate orthogonally protected Fmoc-Cys(Mmt)-OH at the end of the Ado linker and coupled using standard NR-Fmoc/tBu strategy to give intermediate 7. The amine group of Cys was then chosen to couple DOTA on-resin using DOTA-NHS ester (2 equiv) and iPr2NEt (8 equiv) in DMF for overnight to give 8. Note that commercially available DOTA-NHS (Macrocyclics, TX) contains an estimated 3 equiv of TFA by weight. Also, DOTA can alternatively be coupled using a maleimide derivative, following selective cleavage of Mmt group with 3% TFA, 5% iPr3SiH in CH2Cl2. The peptide was then cleaved from the resin, and europium chelation was carried out in solution to yield ligand 2. The purified compound was dissolved in (NH4)2CO3/NH4OAc buffer (pH 8) and 3 equiv of EuCl3.6H2O was added. The reaction stirred overnight in inert atmosphere (to prevent disulfide formation by air oxidation). The excess salts were then removed by solid-phase extraction (SPE). The purified peptides were dissolved in DMSO/H2O (3:2) for bioassay purposes. No diketopiperazine formation was observed in this solvent system for 1 month. (Carpenter, K. A.; Weltrowska, G.; Wilkes, B. C.; Schmidt, R.; Schiller, P. W. J. Am. Chem. Soc. 1994, 116, 8450-8458)

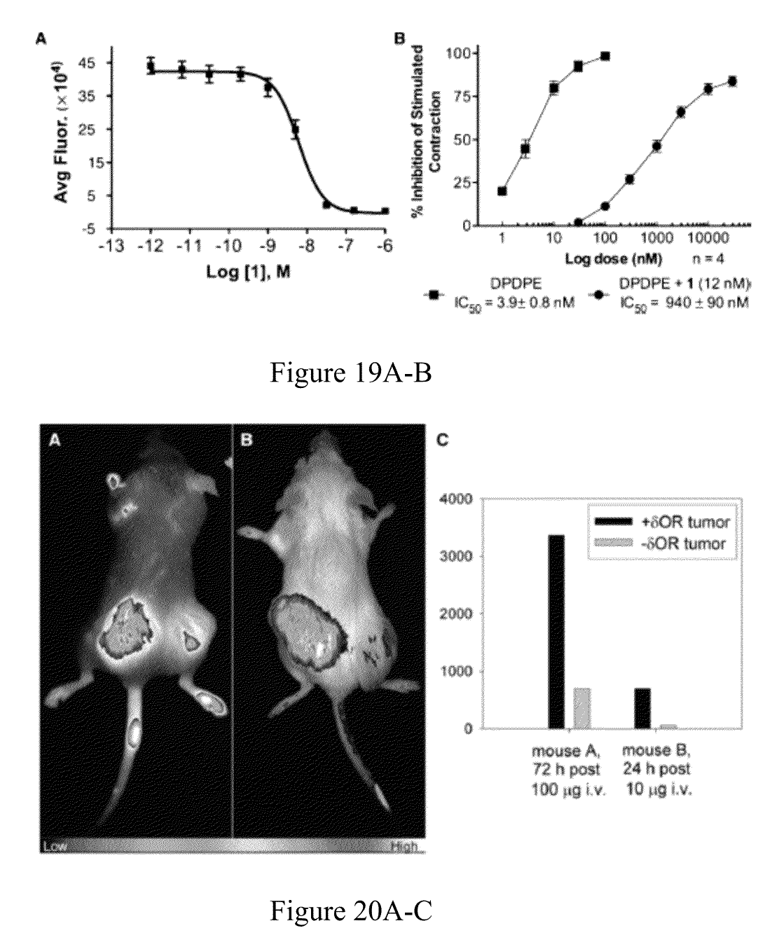

Purified ligands 1 and 6 were evaluated for their binding affinity for the δOR in a competitive binding assay using HCT116 colon carcinoma cells engineered to express the δOR (FIG. 19). Europium-labeled opioid peptide DPLCE was used as a competing ligand in a time-resolved fluorescence (TRF) assay of our own design. (Handl, H. L.; Vagner, J.; Yamamura, H. I.; Hruby, V. J.; Gillies, R. J. Anal. Biochem. 2005, 343, 299-307; Handl, H. L.; Vagner, J.; Yamamura, H. I.; Hruby, V. J.; Gillies, R. J. Anal. Biochem. 2004, 330, 242-250)

Peptide 6 retained high δ receptor affinity (Ki) 2.5 (0.8 nM), which was (Ki) 0.71 nM) and significantly lower than for Dmt-Tic-Lys(Ac)-OH (Ki) 0.047 nM). (Villemagne, P. S.; Dannals, R. F.; Ravert, H. T.; Frost, J. J. Eur. J. Nucl. Med. Mol. Imaging 2002, 29, 1385-1388; Pol, O.; Palacio, J. R.; Puig, M. M. J. Pharmacol. Exp. Ther. 2003, 306, 455-462) However, a direct comparison between these TRF results and reported radioligand binding assays is not wholly instructive because of inherent difference in the assay methods. Ligand 1, with a Cy5 label, exhibited a similar bioactivity profile as ligand 6 and retained high δOR affinity (Ki) 3 (0.1 nM), which is equipotent to its unlabeled counterpart. Thus, attachment of the Mpr spacer and the Cy5 label did not interfere in any significant way in the ligand-receptor interaction. This is in sharp contrast to many labeled opioid peptides (including Tic-based analogues) with remarkably high loss in affinity for δOR. (Arttamangkul, S.; Alvarez-Maubecin, V.; Thomas, G.; Williams, J. T.; Grandy, D. K. Mol. Pharmacol. 2000, 58, 1570-1580)

Further, ligand 1 exhibited high inhibitory potency (Ke) 37 (9 pM for n) 8) in the mouse-isolated vas deferens (MVD) assay against δ-agonist DPDPE (FIG. 19B), which clearly demonstrates it as one of the best labeled δOR ligands. (Kramer, T. H.; Davis, P.; Hruby, V. J.; Burks, T. F.; Porreca, F. J. Pharmacol. Exp. Ther. 1993, 266, 577-584)

For in vivo studies, SCID mice were xenografted bilaterally with HCT116/δOR and parental HCT116 tumors, which do not express δOR. Mice then were tail vein injected with 10 μg of ligand 1 and images were acquired at different times post-injection using a VersArray 1300B cooled CCD camera, a filtered fiberoptic light source and a tunable emission filter (CRI, Inc.). After 15 min post-injection, the fluorescence intensity maps indicated systemic presence of the compound with high intensity throughout the animal (not shown). At 24 h, the compound was systemically cleared and retained in the δOR-containing but not the parental tumors, as shown in FIGS. 20A and B. Images were analyzed using Image-Pro Plus 5 by drawing regions-of-interest (ROIs) over each tumor and noninvolved muscle tissue. Histograms were generated for each ROI, and mean fluorescence intensities were determined for each time point. After 72 h, all δOR(+) tumors had elevated fluorescence intensities compared to the corresponding δOR(−) tumors (FIG. 20C). Notably, this intensity differential was independent of dose, although higher contrast was observed at low dose (10 μg) after 24 h (cf. high dose (100 μg) animals). The Cy5-labeled opioid peptide provided a high contrast noninvasive molecular marker for live imaging of cultured cells or in vivo imaging.

As shown above, the inventors have described a solid-phase synthetic methodology for derivatization of the highly potent δ-opioid ligand Dmt-Tic-Lys(R)-OH. Easy modification with fluorescent dyes and/or chelating labels either on-resin or in solution was sought. The applicability of this synthetic approach was demonstrated by derivatizing a Dmt-Tic ligand with the lanthanide chelator on a solid-phase support and the Cy5 label in solution. Finally, bioevaluation of the Cy5-labeled compound demonstrated its potential utility in in vitro studies and in vivo imaging of the peripherally expressed δ-opioid receptor.

Dmt-Tic IR800

Synthesis and Characterization of Dmt-Tic-IR800

The inventors have previously described the synthesis of a δOR-targeted fluorescent agent (Dmt-Tic-Lys-Cy5) based on a small synthetic peptide antagonist Dmt-Tic (Dmt: 2′,6′-dimethyl-L-tyrosine; Tic: 1,2,3,4-tetrahydroisoquinoline-3-carboxylic acid) conjugated to a fluorescent dye (Cy5) via a small linker to the side chain of lysine (Lys). (Salvadori S, Attila M, Balboni G, Bianchi C, Bryant S D, Crescenzi O, Guerrini R, Picone D, Tancredi T, Temussi P A. Delta opioidmimetic antagonists: prototypes for designing a new generation of ultraselective opioid peptides. Mol Med. 1995; 1(6):678-89; Josan J S, Morse D L, Xu L, Trissal M, Baggett B, Davis P, Vagner J, Gillies R J, Hruby V J. Solid-Phase Synthetic Strategy and Bioevaluation of a Labeled δ-Opioid Receptor Ligand Dmt-Tic-Lys for In Vivo Imaging. Org Lett. 2009; 11(12):2479-82; Josan J S, De Silva C R, Yoo B, Lynch R M, Pagel M P, Vagner J, Hruby V J. Fluorescent and Lanthanide Labeling for Ligand Screens, Assays, and Imaging. Methods Mol Biol. 2011; 716:89-126) This probe was shown to have high binding affinity for the δOR (Kᵢ=3 nM), high inhibitory potency (Kₑ=37 pM), and good in vivo selectivity, pharmacokinetics and biodistribution profiles. (Josan J S, Morse D L, Xu L, Trissal M, Baggett B, Davis P, Vagner J, Gillies R J, Hruby V J. Solid-Phase Synthetic Strategy and Bioevaluation of a Labeled δ-Opioid Receptor Ligand Dmt-Tic-Lys for In Vivo Imaging. Org Lett. 2009; 11(12):2479-82)

In order to improve the characteristics of the probe for future clinical applications, the inventors synthesized a novel imaging agent using the same targeting moiety (Dmt-Tic) but conjugated it to a longer wavelength near-infrared fluorescent dye, Licor IR800CW. The inventors chose the Licor IR800CW dye for these studies because it has excitation and emission wavelengths above 750 nm resulting in less background due to decreased tissue absorbance and autofluorescence at this wavelength which results in improved tissue penetration. In addition, Licor IR800CW is a derivative of the FDA approved indocyanine green (ICG) fluorescent dye, has been shown to be non-toxic, and is available with GMP quality enabling an easier transition to the clinic. (Marshall M V, Draney D, Sevick-Muraca E M, Olive D M. Single-Dose Intravenous Toxicity Study of IRDye800CW in Sprague-Dawley Rats. Mol Imaging Biol. 2010; 12:583-94)

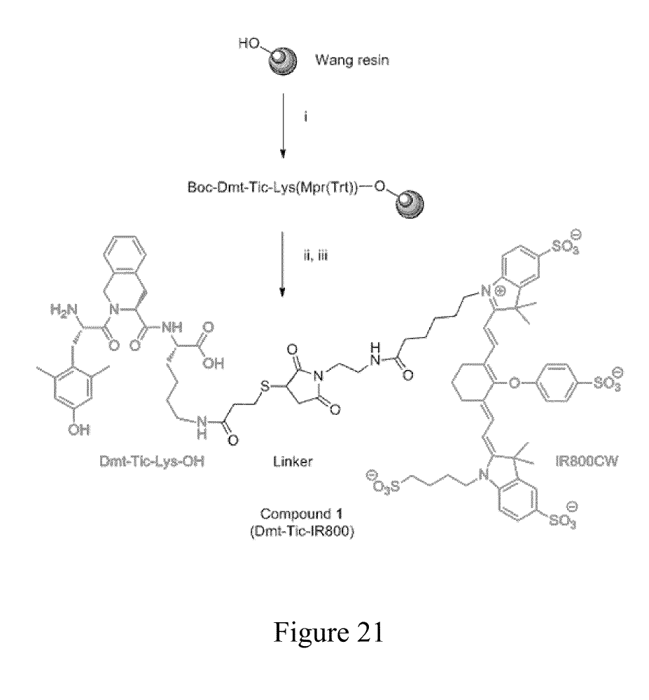

The inventors synthesized Dmt-Tic-IR800 using a synthetic strategy similar to the one described previously (Scheme 4 shown in FIG. 21). (Josan J S, Morse D L, Xu L, Trissal M, Baggett B, Davis P, Vagner J, Gillies R J, Hruby V J. Solid-Phase Synthetic Strategy and Bioevaluation of a Labeled δ-Opioid Receptor Ligand Dmt-Tic-Lys for In Vivo Imaging. Org Lett. 2009; 11(12):2479-82; Josan J S, De Silva C R, Yoo B, Lynch R M, Pagel M P, Vagner J, Hruby V J. Fluorescent and Lanthanide Labeling for Ligand Screens, Assays, and Imaging. Methods Mol Biol. 2011; 716:89-126) The inventors used the same attachment point and linker as in the previously described Dmt-Tic-Lys-Cy5 agent since they were shown not to interfere with binding affinity or selectivity for the δOR. The peptide, Dmt-Tic-Lys-OH, was synthesized on solid phase according to the published procedure. A 3-mercaptopropionyl (Mpr) linker was conjugated to the side chain of the lysine residue of the peptide in order to enable conjugation of the IR800CW dye via thiolmaleimide chemistry. Following cleavage of the peptide-linker conjugate from the resin and purification, the dye conjugation was carried out in solution to afford the final product (Compound 1, Dmt-Tic-IR800, Scheme 4, FIG. 21).

As described above, the inventors have shown that Dmt-Tic binds with high affinity to the δOR and this binding affinity is retained upon conjugation to a fluorescent dye (Cy5). (Josan J S, Morse D L, Xu L, Trissal M, Baggett B, Davis P, Vagner J, Gillies R J, Hruby V J. Solid-Phase Synthetic Strategy and Bioevaluation of a Labeled δ-Opioid Receptor Ligand Dmt-Tic-Lys for In Vivo Imaging. Org Lett. 2009; 11(12):2479-82) To test the binding of Dmt-Tic-IR800 to the δOR, the inventors performed lanthanide time-resolved fluorescence (LTRF) competition binding assays. (Handl H L, Vagner J, Yamamura H I, Hruby V J, Gillies R J. Lanthanide-based time-resolved fluorescence of in cyto ligand-receptor interactions. Anal Biochem. 2004; 330(2):242-50; Handl H L, Vagner J, Yamamura H I, Hruby V J, Gillies R J. Development of a lanthanide-based assay for detection of receptor-ligand interactions at the δ-opioid receptor. Anal Biochem. 2005; 343(2):299-307) This assay uses whole cells, rather than membrane preparations, and thus the only receptors available for binding are those present on the cell surface.

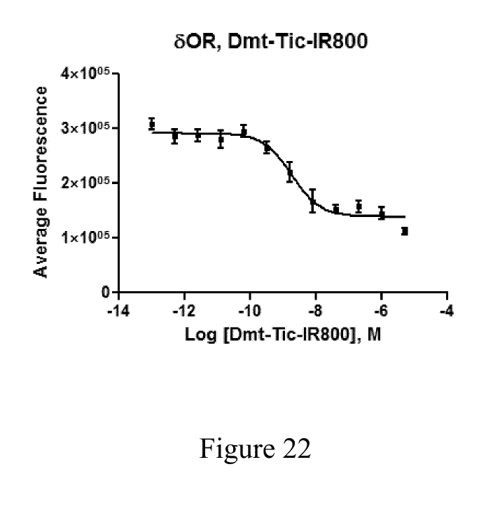

The inventors have previously generated an engineered cell line that stably overexpresses the δO R based on the colorectal cancer cell line HCT-116. (Black K C, Kirkpatrick N D, Troutman T S, Xu L, Vagner J, Gillies R J, Barton J K, Utzinger U, Romanowski M. Gold Nanorods Targeted to Delta Opioid Receptor: Plasmon-Resonant Contrast and Photothermal Agents. Mol Imaging. 2008; 7(1):50-7) The inventors have demonstrated that the parental HCT-116 cell line lacks endogenous expression of the δOR and thus does not bind to the δOR-targeted ligands. In addition, using in cyto time-resolved fluorescence saturation binding assays, the inventors have determined the receptor number for the HCT-116/δOR engineered cell line to be 1.61×10⁶±1.07×10⁵ δOR/cell. For the LTRF competition binding assays with Dmt-Tic-IR800, the inventors used the engineered HCT-116/δOR cell line since it has a high number of receptors on the cell surface. Cells were incubated with europium-labeled delta opioid receptor agonist (Eu-DTPA-DPLCE) and increasing concentrations of Dmt-Tic-IR800 as the competing ligand. Higher concentrations of Dmt-Tic-IR800 result in a decrease in signal from the europium-labeled ligand due to competition for binding to the δOR (FIG. 22). An average Kᵢ of 1.43±0.24 nM was obtained, which is similar to that of the Dmt-Tic-Lys-Cy5 ligand, thus verifying that Dmt-Tic-IR800 retains high δOR binding affinity.

Characterization of δOR Expression in Cell Lines

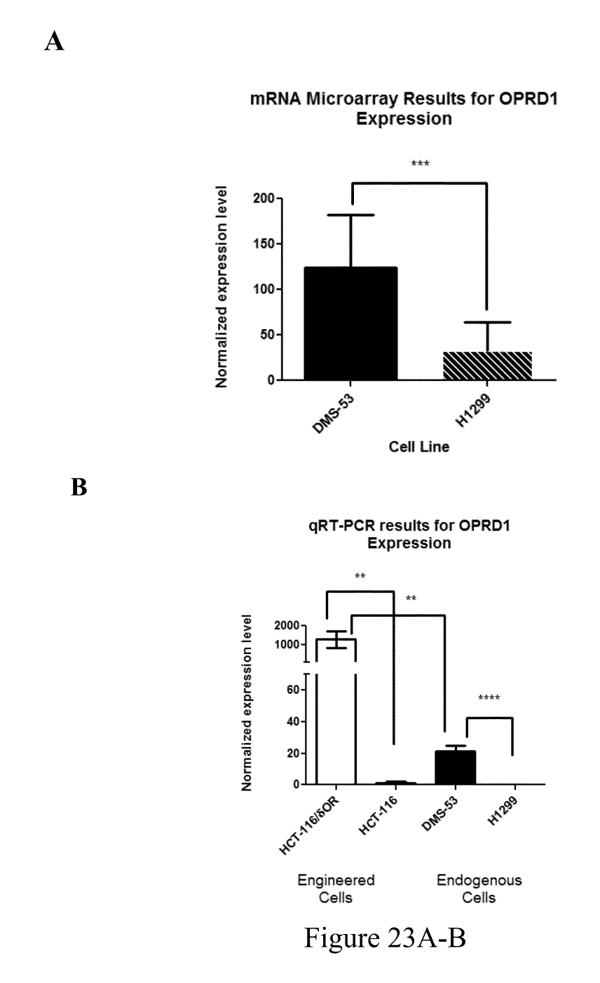

In order to study the δOR-targeted imaging agent, the inventors identified δOR-expressing cell lines that could be used for in vitro and in vivo experiments. As described above, the inventors have previously generated an engineered cell line that stably overexpresses the δOR, HCT-116/δOR. (Black K C, Kirkpatrick N D, Troutman T S, Xu L, Vagner J, Gillies R J, Barton J K, Utzinger U, Romanowski M. Gold Nanorods Targeted to Delta Opioid Receptor: Plasmon-Resonant Contrast and Photothermal Agents. Mol Imaging. 2008; 7(1):50-7) This engineered cell line is useful as a model system since the cells are adherent and are capable of forming xenografts in mice. In order to design lung cancer targeted imaging agents, the inventors identified a lung cancer cell line that contains these characteristics and has endogenous expression of the δOR. The inventors analyzed mRNA microarray data for the expression of δOR in a panel of lung cancer cell lines (data not shown). From this screen, the inventors identified DMS-53, a small cell carcinoma cell line, as having high mRNA expression and H1299, a large cell neuroendocrine cell line, as having low/no mRNA expression (FIG. 23A). To further confirm these results, the inventors screened these two lung cancer cell lines using quantitative real-time reverse transcriptase polymerase chain reaction (qRT-PCR) for expression of OPRD1, the gene that encodes the δOR. The inventors also performed qRT-PCR on the parental and δOR-expressing HCT-116 cell lines as negative and positive controls, respectively. In agreement with the microarray data, the inventors found DMS-53 as positive for expression of OPRD1 and H1299 as negative (FIG. 23B). In addition, the inventors compared the expression of OPRD1 in the endogenously expressing DMS-53 cells to that in the engineered HCT-116/δOR cells. As expected, DMS-53 cells have significantly lower expression (˜60 fold) than HCT-116/δOR cells.

Since mRNA expression does not necessarily reflect the protein expression levels, the inventors determined the receptor number for the endogenous lung cancer cell lines. Previously, the inventors have used LTRF saturation binding assays to determine receptor number. (Tafreshi N K, Huang X, Moberg V E, Barkey N M, Sondak V K, Tian H, Morse D L, Vagner J. Synthesis and Characterization of a Melanoma-Targeted Fluorescence Imaging Probe by Conjugation of a Melanocortin 1 Receptor (MC1R) Specific Ligand. Bioconjug Chem. 2012; 23(12):2451-9) However, this technique has a detection limit of approximately ˜10,000 receptors/cell. Attempts to quantify the receptor number on the δOR-expressing DMS-53 cells using this technique were unsuccessful (data not shown). Thus it is concluded that these cells express less than 10,000 receptors.

In Vivo Tumor Targeting

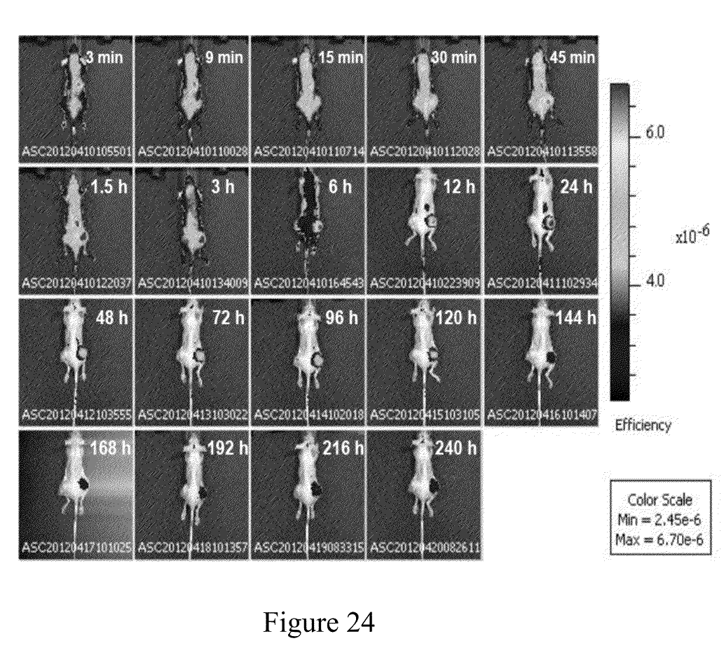

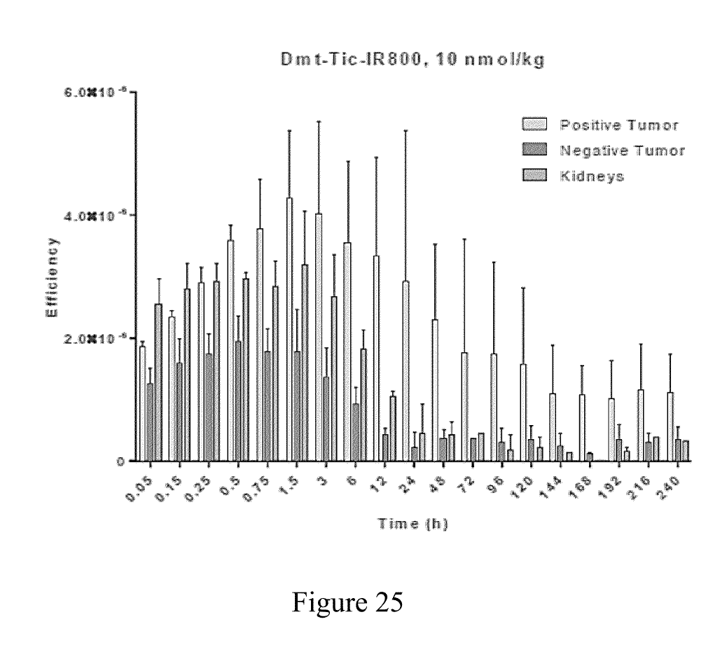

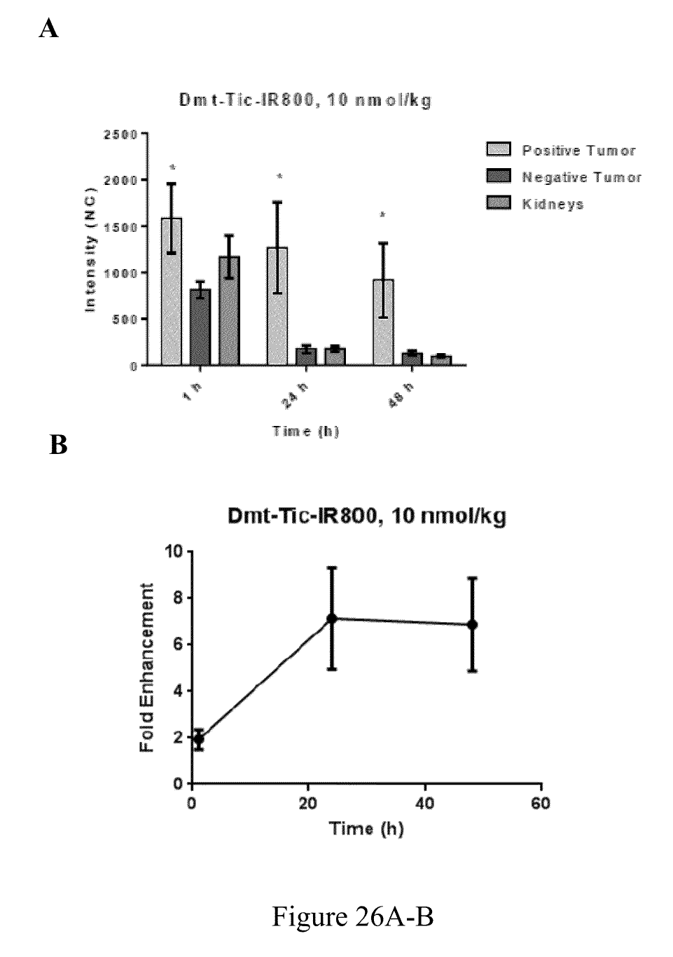

Having demonstrated that Dmt-Tic-IR800 binds to the δOR in vitro, the probe's selectivity was tested in vivo. Initial experiments were performed using the HCT-116 parental cell line and HCT-116/δOR engineered cells in a bilateral flank subcutaneous xenograft model in mice. Previous experiments using these cell lines and the Dmt-Tic-Lys-Cy5 agent showed selective uptake in the HCT-116/δOR tumor at the lowest reliably detectable dose of 4.5 nmol/kg. Due to the longer excitation and emission wavelengths of Dmt-Tic-IR800 in comparison to Dmt-Tic-Lys-Cy5, fluorescence imaging with this probe does not have as much background signal because of lack of tissue autofluorescence. Using the low dose for in vivo detection of Dmt-Tic-Lys-Cy5 as a starting point, the inventors selected doses of Dmt-Tic-IR800 that were lower (2.5 nmol/kg), equivalent (5 nmol/kg), and higher (10 and 20 nmol/kg) than that used in the prior studies. Mice were injected intravenously with the designated concentrations of Dmt-Tic-IR800 and fluorescence imaging was performed at various time points post-injection using the IVIS200 imaging system. The data were analyzed for differences in the intensity of the signal with the various concentrations and also for selectivity of the ligand for the target-expressing relative to the control tumors (fold of enhancement). An optimized dose of 10 nmol/kg Dmt-Tic-IR800 was selected. FIG. 24 depicts the sequence of representative in vivo fluorescence images following administration of the optimized dose at different time points showing selective tumor and systemic uptake of the agent and systemic clearance via the kidney. FIG. 25 depicts the pharmacokinetics of uptake and clearance in the positive tumor (engineered cells), negative tumor (parental cells) and kidneys by quantification of fluorescence signal at each time point. FIG. 26 (A) depicts the quantified in vivo fluorescence imaging values for the positive and negative tumors and kidneys at the 1, 24 and 48 h time points, and (B) which depicts the fold enhancement of the fluorescence signal in the positive tumor relative to the negative tumor at the same time-points.

Using this optimized dose, the inventors performed another experiment on mice bearing HCT-116 cells in the left flank and HCT-116/δOR cells in the right flank. The mice were imaged at 24 hours post-injection as this time point shows maximum fold of enhancement between the positive and negative tumors. The mice were imaged using the Optix MX3 fluorescence imaging system. This system uses a pulsed laser, raster scanning illumination and collection, and a time-correlated single photon counting system instead of the epi-illumination and cooled charge coupled device camera found on the IVIS200.

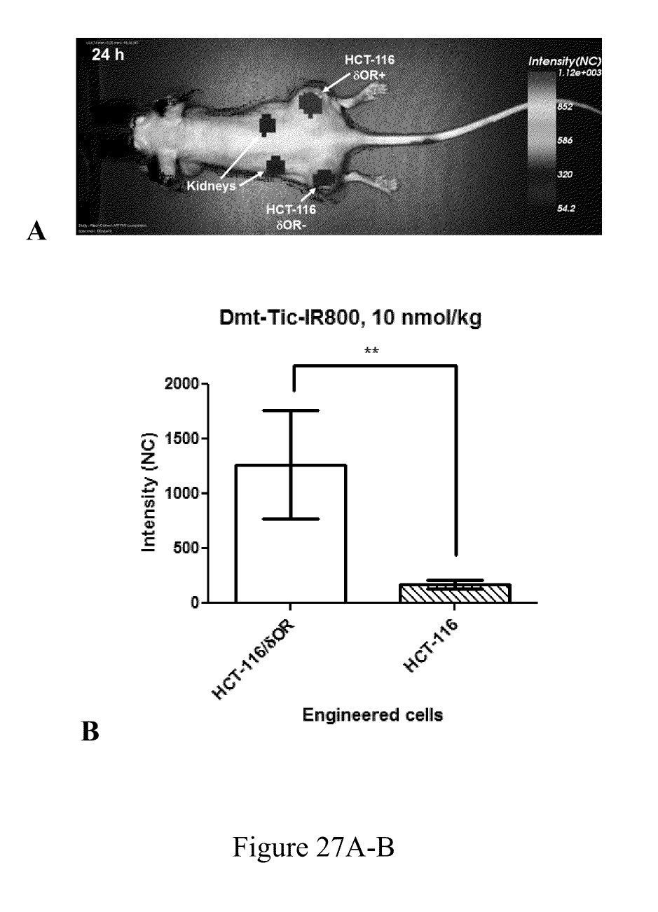

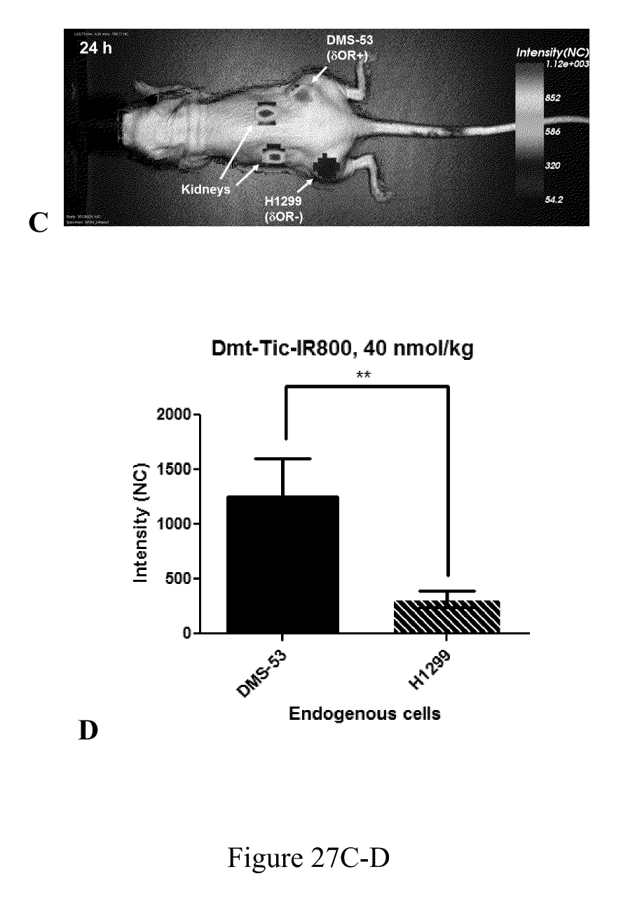

The HCT-116/δOR tumors had significantly higher average fluorescence signal than the HCT-116 tumors at 24 hours post-injection of Dmt-Tic-IR800 (P<0.01, n=4) (FIGS. 27A and 27B). The fold enhancement of the positive tumor (HCT-116/δOR) relative to the negative tumor (HCT-116) is ˜7-fold. These results indicate that Dmt-Tic-IR800 is selective for the δOR in vivo.

Since the δOR has significantly higher expression in the engineered cell line as compared to the endogenously expressing lung cancer cell lines (see above), the inventors explored whether Dmt-Tic-IR800 would possess enough sensitivity to detect the lung cancer cell lines in vivo. Mice were subcutaneously injected with H1299 (δOR−) (12 million cells of 100 uL per injection) and DMS-53 (δOR+) (20 million cells at 100 uL per injection) cells in the left and right flanks, respectively. Tumors were grown for 3 weeks after subcutaneous injection.

A dose determination assay was performed to find the optimal concentration of Dmt-Tic-IR800 for further experiments. A higher concentration of the imaging agent in these experiments was needed due to the lower receptor number. Based on previous work with fluorescent imaging agents targeting different receptors in endogenous expressing cell lines, the inventors selected concentrations of 40 nmol/kg, 80 nmol/kg, and 160 nmol/kg. (Huynh A S, Chung W J, Cho H I, Moberg V E, Celis E, Morse D L, Vagner J. Novel Toll-like Receptor 2 Ligands for Targeted Pancreatic Cancer Imaging and Immunotherapy. J Med Chem. 2012; 55(22):9751-62) Fluorescence imaging was performed using the IVIS200 imaging system at various time points following intravenous administration of Dmt-Tic-IR800. For the bilateral flank subcutaneous xenograft model using endogenously expressing cells the optimal dose was 40 nmol/kg.

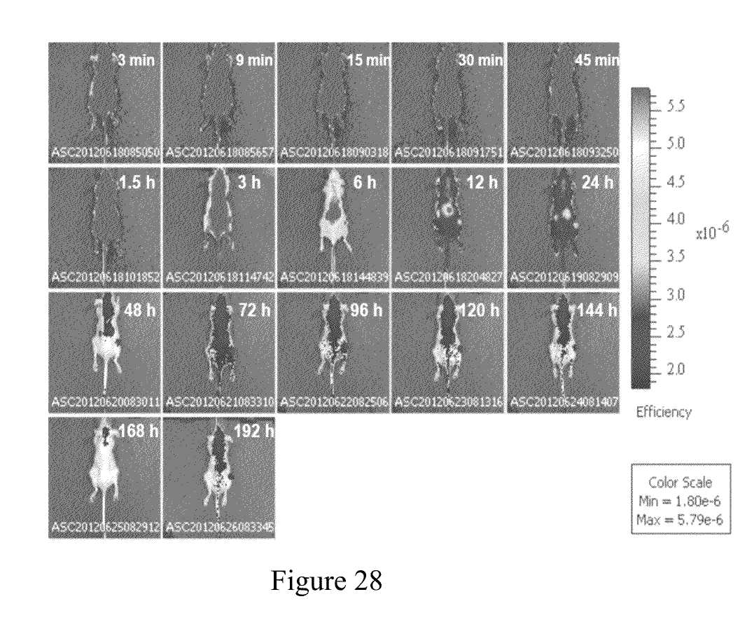

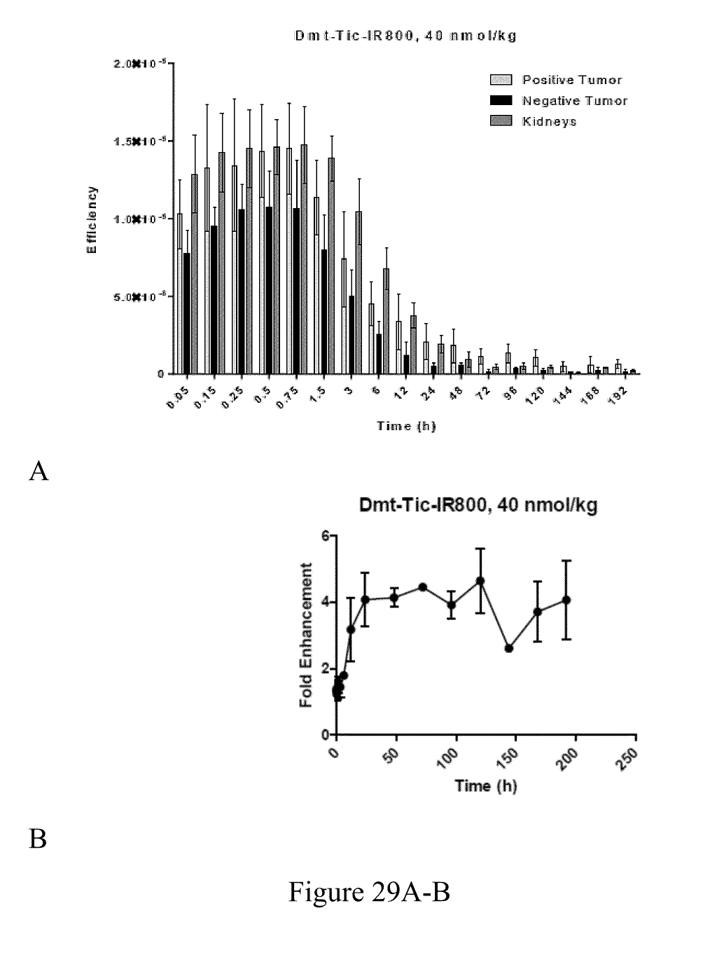

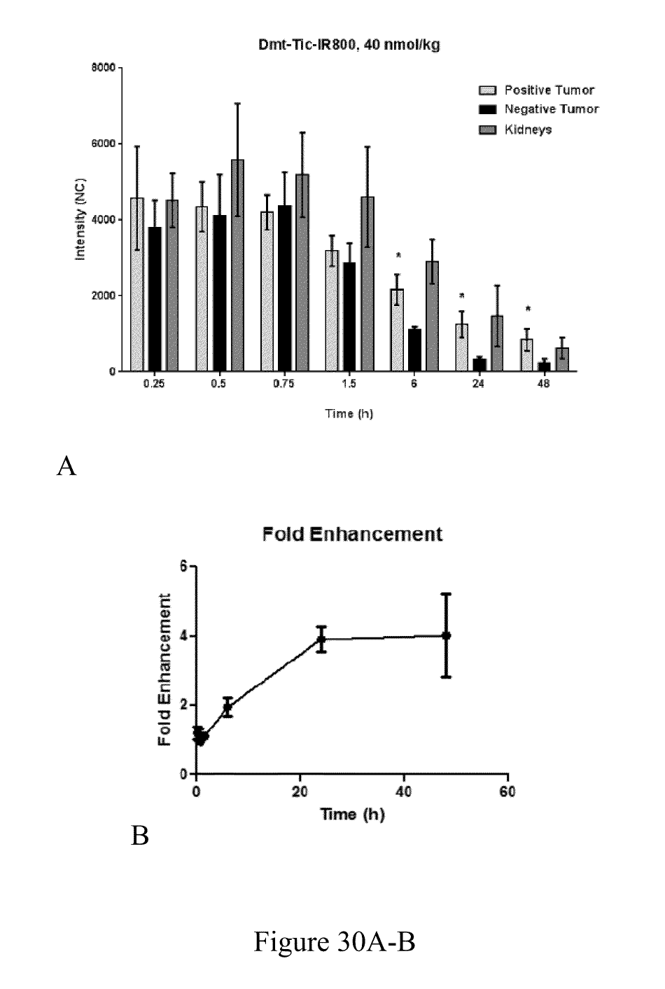

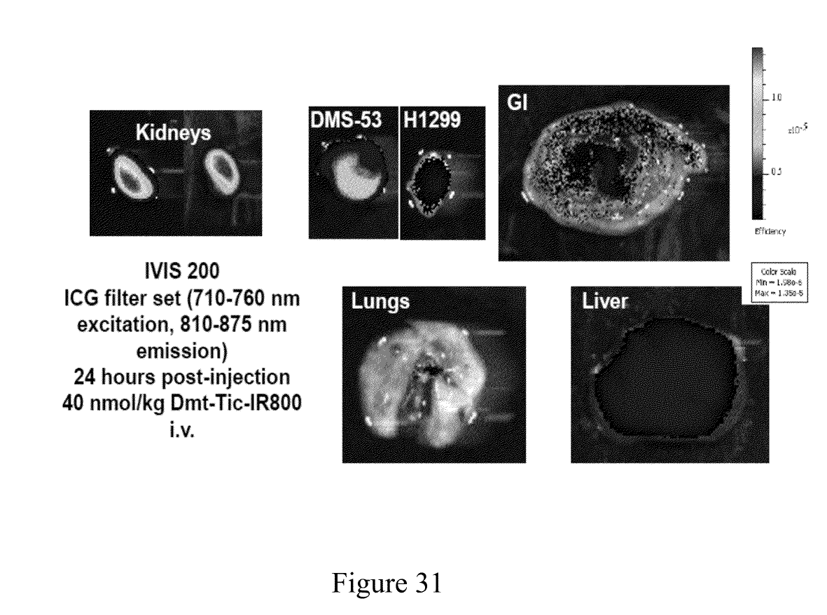

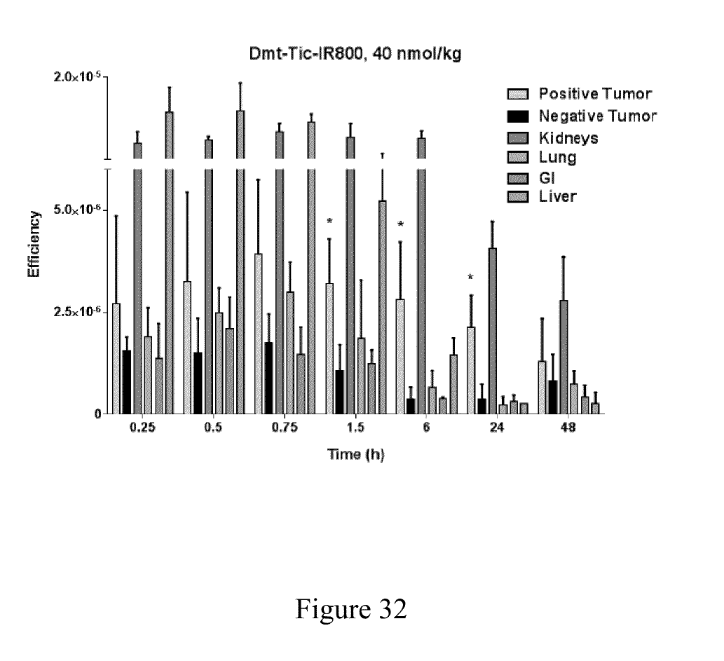

Fluorescence imaging of mice bearing H1299 (δOR−) cells in the left flank and DMS-53 (δOR+) cells in the right flank was performed using the Optix MX3 imaging system and a 40 nmol/kg dose of Dmt-Tic-IR800. At 24 hours post-injection of agent, the DMS-53 (δOR+) cells had significantly higher normalized intensity than the H1299 (δOR−) cells (FIGS. 27C and 27D). The fold enhancement of the positive tumor (DMS-53) relative to the negative tumor (H1299) is ˜4-fold. By the Rose criterion, imaging agents must have a 3-fold enhancement in detection in order to show clinical utility. Thus, by this criterion, Dmt-Tic-IR800 has sufficient sensitivity to detect endogenously expressing lung cancers and potential for use in clinical settings. FIG. 28 is a series of in vivo fluorescence images taken at different time points of mice bearing the xenograft tumors of lung cancer cell lines with positive endogenous expression (DMS-53) and non-expression (H1299). These representative images show the tumor and systemic uptake and clearance of the Dmt-Tic-IR800 agent over a time course. FIG. 29 (A) is a graph of quantified fluorescence intensity values acquired using the IVIS 200 instrument for the tumors and kidneys that depict the pharmacokinetics of tumor and systemic uptake and clearance following intravenous administration of 40 nmol/kg of Dmt-Tic-IR800, and (B) shows the fold enhancement in the positive tumor over the same time-course. FIG. 30 is a series of graphs depicting the quantified values from in vivo imaging of the lung cancer xenograft tumors using the Optix MX-3 imaging instrument by ART. FIG. 31 is a series of representative images depicting the ex vivo fluorescence imaging of excised positive tumor, negative tumor, liver, lungs, kidneys and GI tract following intravenous administration of the Dmt-Tic-IR800 agent. FIG. 32 is a graph depicting the biodistribution in lung cancer xenografts derived from the ex vivo imaging demonstrated in FIG. 31.

Orthotopic Model of Lung Cancer

As discussed above, the inventors have developed an imaging agent for a reported lung cancer marker, the delta opioid receptor (DOR), based on a synthetic peptide antagonist that targets this receptor (Dmt-Tic) conjugated to a fluorescent dye IRDye800CW. The inventors evaluated this novel agent, DORL-800, for imaging of the DOR both in vitro and in vivo. DOR protein expression was verified in patient samples using immunohistochemistry (IHC) of a lung cancer tissue microarray (TMA). By competitive binding assay, DORL-800 was demonstrated to have high affinity for the DOR in vitro=1.43±0.24 nM, n=3). The pharmacokinetics (PK) and biodistribution (BD) of DORL-800 was evaluated in a bilateral subcutaneous xenograft model using endogenously expressing lung cancer cell lines, DMS-53 (DOR+) and H1299 (DOR−). DORL-800 can be used for fluorescence imaging in this model.

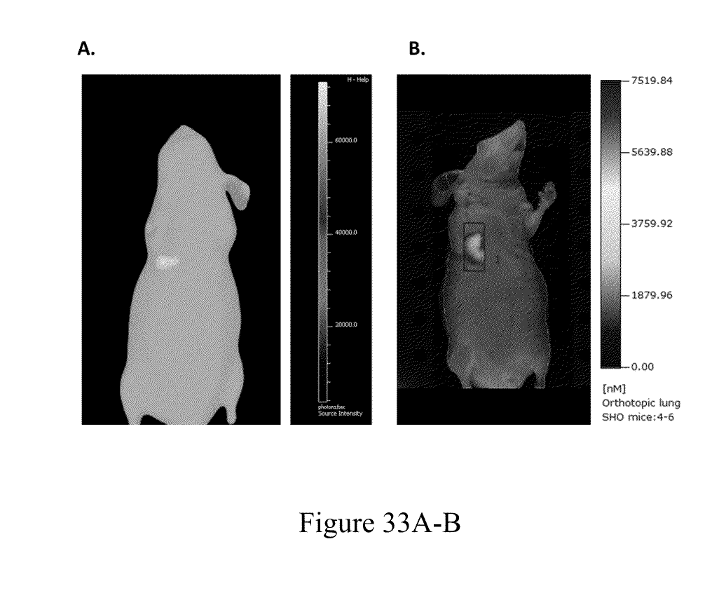

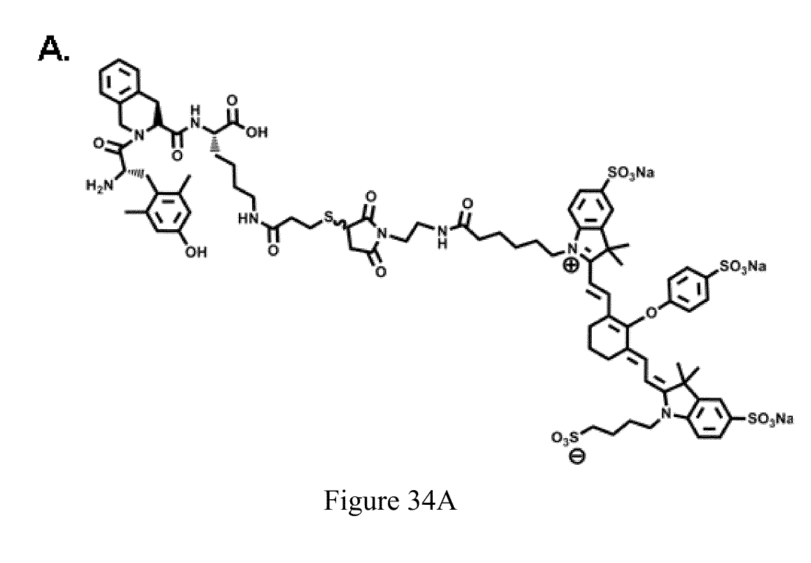

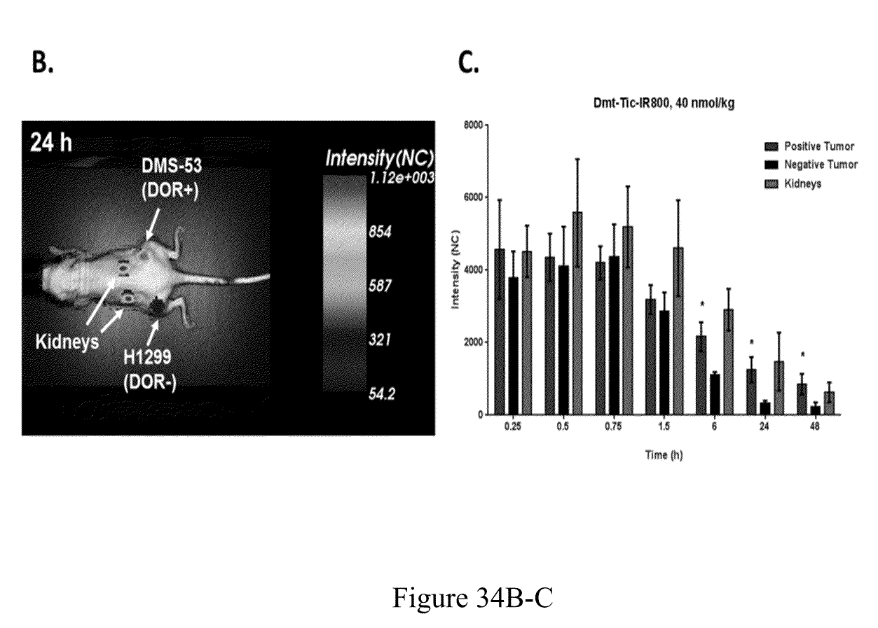

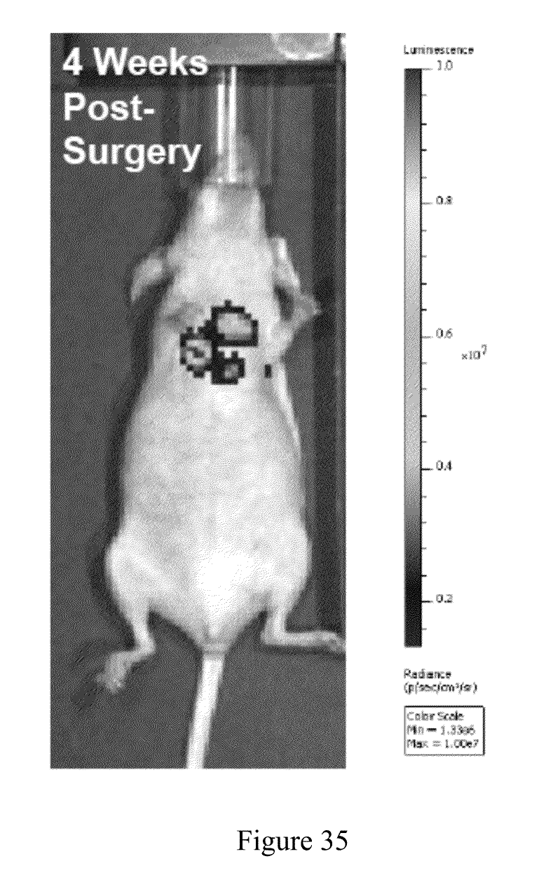

In the orthotopic model of lung cancer, the mice undergo a minor surgical procedure during which DMS-53 luc+ (DOR+) cells are injected directly in the left lung. The growth of the tumors is monitored using bioluminescence imaging (FIG. 33A). The PK of DORL-800 was studied in this model by injecting mice intravenously with DORL-800 (40 nmol/kg) and acquiring images at various time points from 0 to 48 hours using fluorescence molecular tomography (FIG. 33B). DORL-800 accumulated specifically in the tumor. FIG. 34A depicts the structure of DORL-800. FIGS. 34B and C depict in vivo fluorescence imaging of endogenously expressing subcutaneous lung cancer xenograft tumors following administration of DORL-800. The presence of tumor was confirmed by computed tomography (CT) and histology. DORL-800 was studied for use in intraoperative guidance. FIG. 35 is an image of a SCID Hairless Outbred mouse (SHO) four weeks after DMS-53 luc+ (DOR+) cells were injected directly into the lung.

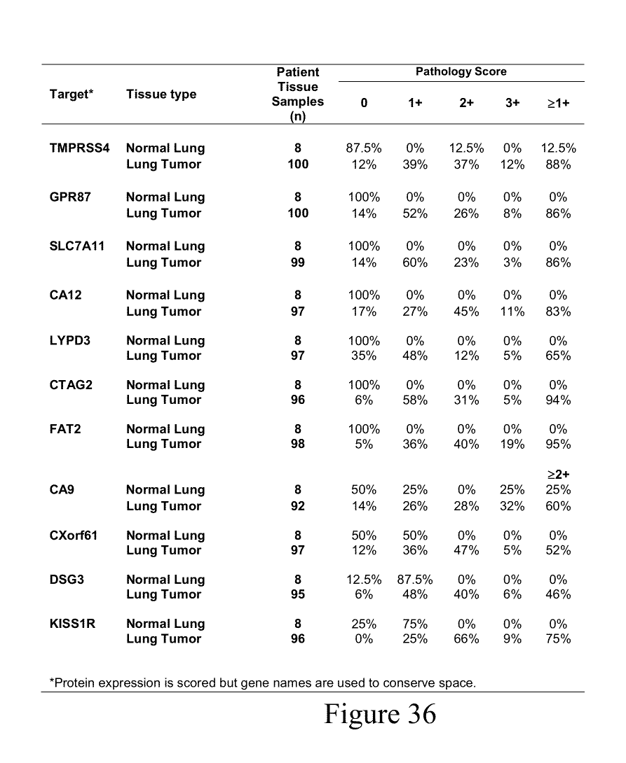

Microarray data from patient samples were used for the identification of additional cell-surface markers for lung cancer. Eleven new cell-surface markers were identified based on increased expression in lung tumors over non-neoplastic lung samples and limited expression in other organs. The inventors validated protein expression for these markers using IHC of the lung cancer TMA and also found that high expression of several of the markers corresponds to shorter survival. (FIG. 36)

Materials and Methods

Probe Synthesis, Purification and Characterization

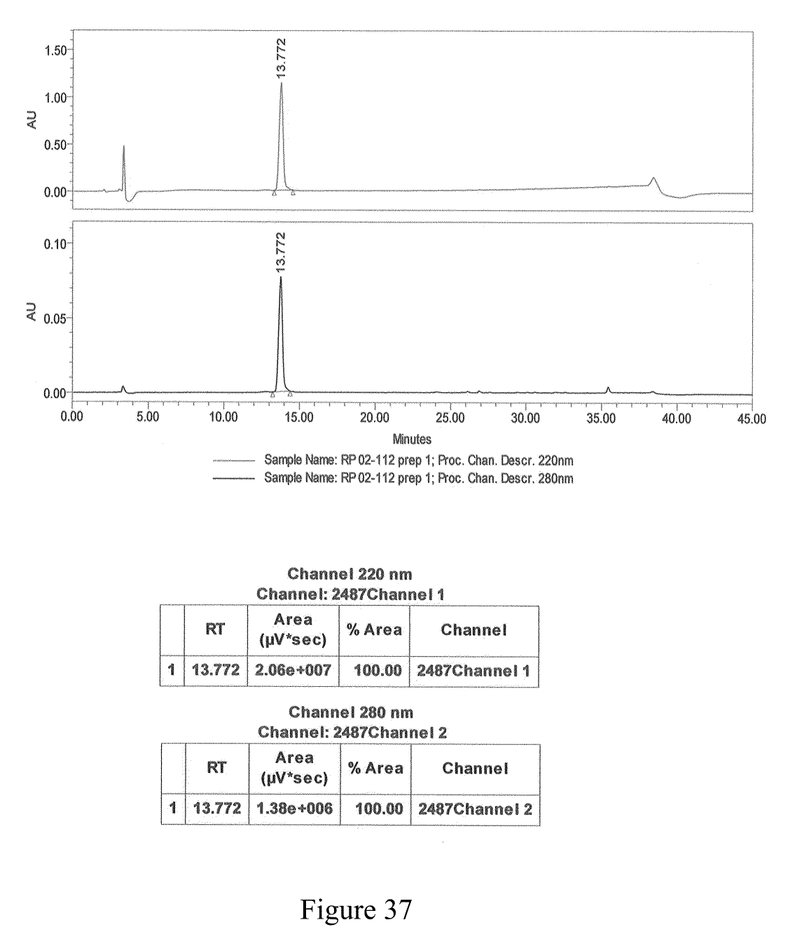

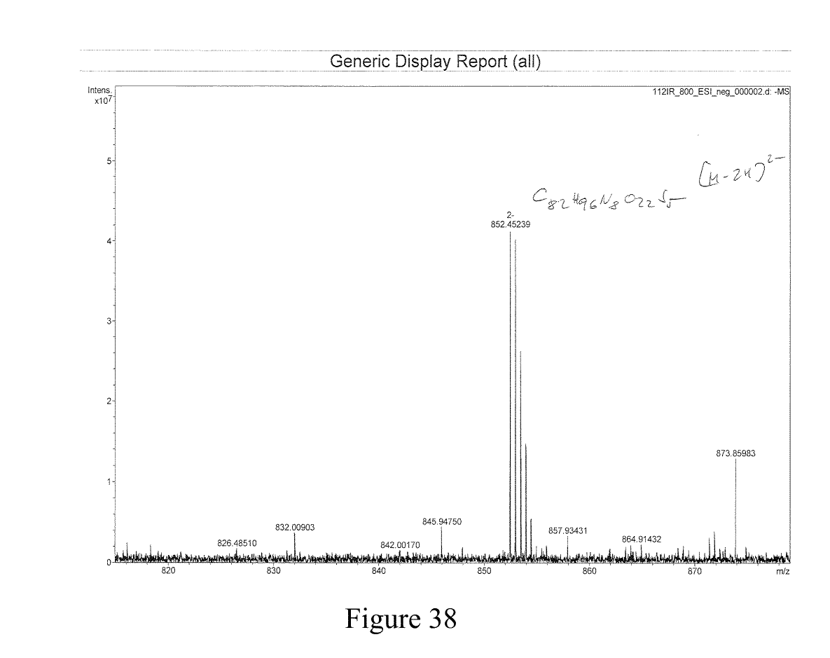

Labeled analog Dmt-Tic IR800, compound 1, was synthesized using standard Fmoc chemistry on Wang resin as described in Scheme 4 and in detail in Josan, et al. (Josan J S, Morse D L, Xu L, Trissal M, Baggett B, Davis P, Vagner J, Gillies R J, Hruby V J. Solid-Phase Synthetic Strategy and Bioevaluation of a Labeled δ-Opioid Receptor Ligand Dmt-Tic-Lys for In Vivo Imaging. Org Lett. 2009; 11(12):2479-82; Josan J S, De Silva C R, Yoo B, Lynch R M, Pagel M P, Vagner J, Hruby V J. Fluorescent and Lanthanide Labeling for Ligand Screens, Assays, and Imaging. Methods Mol Biol. 2011; 716:89-126) Briefly, the peptide Dmt-Tic-Lys(Mpr)-OH was assembled on the solid support and cleaved from the resin by treatment with a TFA-scavenger cocktail consisting of trifluoroacetic acid (TFA) (91%), water (3%), triisopropylsilane (3%), and thioanisole (3%) for 3 hours. The peptide was purified by reverse phase high performance liquid chromatography (RP-HPLC) on a Waters 600 HPLC using a reverse phase C18 column (Vydac C18, 15-20 μm, 22×250 mm). The peptide intermediate was eluted with a linear gradient of acetonitrile (CH₃CN)/0.1% TFA (CF₃CO₂H) at a flow rate of 5.0 mL/min. The intermediate was dissolved in dimethyl sulfoxide (DMSO) and treated with 1.3 eq IRDye® 800CW maleimide (Li-cor Biosciences, Lincoln, Nebr.) for 16 hours. The reaction mixture was diluted with water and loaded to the C-18 Sep-Pak™ cartridge (100 mg, Waters, Milford, Mass.). The cartridge was washed with deionized (DI) water, and then gradually with 5%, 10%, 60%, and 90% aqueous acetonitrile (CH₃CN) to elute the ligand. The purity (>99%) of compound 1 was determined by analytical RP-HPLC using a Waters Alliance 2695 Separation Model with a Waters 2487 dual wavelength detector (220 and 280 nm) on a reverse phase column (Waters Symmetry C18, 3.0×75 mm, 3.5 μm). The compound showed an elution time of 13.77 minutes with a linear gradient of 10%-90% aqueous CH₃CN/0.1% CF₃CO₂H at a flow rate of 0.3 mL/min (FIG. 37). Electro-spray ionization-mass spectrometry (ESI-MS) in negative mode confirmed the structure of compound 1 [(M-2H)²⁻ calc. 852.7667. found 852.524] (FIG. 38).

Cell Culture

HCT-116 and DMS-53 cells were obtained from the ATCC (American Type Culture Collection, Manassas, Va.). H1299 cells were kindly provided by the Lung SPORE cell line repository at H. Lee Moffitt Cancer Center & Research Institute. HCT-116/δOR cells were previously generated using pcDNA-δOR15 vector containing a truncated δOR lacking the final 15 C-terminal amino acids. (Black K C, Kirkpatrick N D, Troutman T S, Xu L, Vagner J, Gillies R J, Barton J K, Utzinger U, Romanowski M. Gold Nanorods Targeted to Delta Opioid Receptor: Plasmon-Resonant Contrast and Photothermal Agents. Mol Imaging. 2008; 7(1):50-7) HCT-116 and HCT-116/δOR cells were cultured in DMEM/F-12 (1:1) media containing 365 mg/L L-Glutamine, 2.438 g/L Sodium Bicarbonate (Life Technologies, Gibco), 10% fetal bovine serum (Atlanta Biologicals), 100 units/mL penicillin, and 100 μg/mL streptomycin. H1299 cells were cultured in RPMI-1640 media containing 300 mg/L L-Glutamine (Life Technologies, Invitrogen), 10% fetal bovine serum (Atlanta Biologicals), 100 units/mL penicillin, and 100 μg/mL streptomycin. DMS-53 cells were cultured in RPMI-1640 media containing 300 mg/L L-Glutamine (Life Technologies, Invitrogen) and 10% fetal bovine serum (Atlanta Biologicals). The cells were incubated in 5% CO₂ at 37° C. The morphology and growth characteristics of these cells were monitored throughout by microscopy.

Characterization of δOR Expression in Cell Lines by DNA Microarray Expression Profiling

All the data was selected because the arrays were run on U133 Plus 2.0 arrays from Affymetrix and raw CEL files were available for download. Datasets were identified at the Gene Expression Omnibus at the National Center for Biotechnology Information (NCBI) and ArrayExpress at the European Bioinformatics Institute (EBI) that contained arrays run with lung cancer derived cell lines. The samples used in this analysis were from the accession numbers: GSE5816, GDS2604, GSE5816, GSE4824, GSE7562, GSE8332, GSE10843, GSE13309, GSE14315, GSE14883, GSE15240, GSE16194, GSE17347, GSE18454, GSE21612, and E-MTAB-37.

All CEL files were loaded into the Affymetrix Expression Console software and processed with the MAS 5.0 algorithm to calculate signal intensities using a trimmed mean average of 500 to scale all samples. The quality of individual samples was evaluated from the quality metrics from the Expression Console reports, R QC reports, and an RNA quality analysis tool developed at the Moffitt Cancer Center. Samples were rejected for having high scaling factors (>12), low percent present calls (<35), high RNA quality scores (>4.0), and odd looking scatter plots when compared to a reference array. Individual probes corresponding to the genes of interest (207792 at corresponding to OPRD1) were extracted from the full array data to determine the relative expression of genes in the different cell lines.

Characterization of δOR Expression in Cell Lines by Quantitative Real-Time Reverse Transcriptase Polymerase Chain Reaction (qRT-PCR)

RNA extractions were performed on cell lines using the RNeasy®Mini Kit (Qiagen, Cat. #74104) following the manufacturer's instructions which include the DNase digestion steps. RNA concentration and purity (A₂₆₀/A₂₈₀ ratio) were determined by using the Nanodrop Spectrophotometer, ND-1000. qRT-PCR was performed using the Smart Cycler (Cephid, Sunnyvale, Calif.). δOR specific primer sets were designed using Gene Runner software for Windows v 3.05: forward, 5′-GGTGACCAAGATCTGCGTGTTC-3′ (SEQ ID NO:1) and reverse, 5′-TTCTCCTTGGAGCCCGACAG-3′ (SEQ ID NO:2). The iScript One-Step RT-PCR Kit with SYBR Green (Bio-Rad, Cat. #170-8893) was used for qRT-PCR. During each experiment, reactions were performed using template without RT mix and with no-template added as controls. β-actin (ACTB) was used for normalization. (Morse D L, Carroll D, Weberg L, Borgstrom M C, Ranger-Moore J, Gillies R J. Determining suitable internal standards for mRNA quantification of increasing cancer progression in human breast cells by real-time reverse transcriptase polymerase chain reaction. Anal Biochem. 2005; 342:69-77)

The following conditions for thermocycling were used: Stage 1 was held at 50° C. for 10 min for cDNA synthesis; stage 2 was held at 95° C. for 5 min for reverse transcriptase inactivation; stage 3 cycled 40 times through two temperatures for PCR amplification, starting with 95° C. for 10 sec and Tₘ for 30 sec (Tₘ is 60° C. for ACTB and 62° C. for δOR); and stage 4 included a melt curve for quality control, starting at 40° C. and ending at 95° C. (increasing by 0.2° C. each cycle). Values were calculated as Expression=2^{-Ct(δOR)}/2^{-Ct(ACTB)}×1000. Each experiment was repeated 3 times to determine reproducibility.

Binding Assays on Engineered Cells

To determine binding affinity the inventors used a lanthanide time-resolved fluorescence (LTRF) competitive binding assay as described previously. (Handl H L, Vagner J, Yamamura H I, Hruby V J, Gillies R J. Lanthanide-based time-resolved fluorescence of in cyto ligand-receptor interactions. Anal Biochem. 2004; 330(2):242-50; Handl H L, Vagner J, Yamamura H I, Hruby V J, Gillies R J. Development of a lanthanide-based assay for detection of receptor-ligand interactions at the δ-opioid receptor. Anal Biochem. 2005; 343(2):299-307) HCT-116 colorectal cancer cells engineered to express the δOR (HCT-116/δOR) were used to assess ligand binding. Europium (Eu)-diethylenetriaminepentaacetic acid (DTPA)[D-Pen², L-Cys⁵] enkephalin (DPLCE), a δOR agonist, was used as the competed ligand. (Josan J S, De Silva C R, Yoo B, Lynch R M, Pagel M P, Vagner J, Hruby V J. Fluorescent and Lanthanide Labeling for Ligand Screens, Assays, and Imaging. Methods Mol Biol. 2011; 716:89-126; Handl H L, Vagner J, Yamamura H I, Hruby V J, Gillies R J. Development of a lanthanide-based assay for detection of receptor-ligand interactions at the δ-opioid receptor. Anal Biochem. 2005; 343(2):299-307)

Using in cyto time-resolved fluorescence (TRF) saturation binding assays with Eu-DTPA labeled DPLCE, the inventors have determined the K_{d}, Bₘₐₓ, and receptor number for the HCT-116/δOR cell line to be 51.76 nM, 3,011,000 AFU, and 1.61×10⁶±1.07×10⁵ δOR/cell, respectively. For the competitive binding assays, HCT-116/δOR cells were plated in black wall/white bottom 96-well plates (Perkin Elmer, Cat. #6005060) at a density of 20,000 cells per well and were allowed to grow for 3 days.

On the day of the experiment, media were aspirated from all wells and then the cells were rinsed with phosphate buffered saline (PBS) (100 μL/well). 50 μL of Dmt-Tic-IR800 (dilutions ranging from 1×10⁻⁵ to 2.05×10⁻¹³M) and 50 μL of Eu-DTPA labeled DPLCE (10 nM, K_{d}=51.76 nM) were added to each well. Ligands were diluted in binding assay buffer (Modified Eagles medium [MEM] (Gibco, Cat. #61100-087), 1 mM 1,10-phenanthroline, 200 mg/L bacitracin, 0.5 mg/L leupeptin, 26 mM NaHCO₃, 25 mM HEPES, 0.2% w/v BSA) and samples were tested in octuplicate.

Cells were incubated in the presence of ligands for 1 h at 37° C. and 5% CO₂. Following the incubation, cells were washed three times with wash buffer (50 mM Tris-HCl, 0.2% w/v BSA, 30 mM NaCl) (200 μL/well). DELFIA enhancement solution (Perkin Elmer, Cat. #1244-105) was added (100 μL/well), and plates were incubated for 30 min at room temperature prior to reading. The plates were read on a Perkin Elmer Victor X4 instrument using the standard Eu TRF measurement settings (340 nm excitation, 400 μs delay, and emission collection for 400 μs at 615 nm).

Competition curves were analyzed with GraphPad Prism software using the sigmoidal dose-response (variable slope) classical equation for nonlinear regression analysis. The Kᵢ for Dmt-Tic-IR800 was calculated using the equation Kᵢ=IC₅₀/(1+[ligand]/K_{d}), where IC₅₀ is determined from the competition curves, [ligand] is the final concentration of Eu-DTPA labeled DPLCE (5 nM) and K_{d} is the dissociation constant for Eu-DTPA labeled DPLCE (51.76 nM). The number given is the average Kᵢ obtained from three independent experiments.

Tumor Xenograft Studies and Fluorescence Imaging

All procedures were in compliance with the Guide for the Care and Use of Laboratory Animal Resources (1996), National Research Council, and approved by the Institutional Animal Care and Use Committee, University of South Florida. Female nu/nu mice 6-8 weeks old (Harlan Laboratories, Indianapolis, Ind.) were injected subcutaneously (s.c.) with 8×10⁶ HCT-116/δOR cells in the right flank and the same number of parental cells in the left flank. Tumors were allowed to grow for 2 weeks. Four days prior to imaging the mice were switched to fluorescence imaging feed (AIN 93G). For the dose determination studies, mice were injected with one of four doses of Dmt-Tic-IR800 (20 nmol/kg, 10 nmol/kg, 5 nmol/kg or 2.5 nmol/kg) in 100 μL PBS via the tail vein. Animals were anesthetized using isoflurane (flow 2-2.5 L/min) and were positioned on the heated stage for imaging.

Fluorescence images were acquired pre-injection and at various time points post-injection of the ligand using the Xenogen IVIS-200 imaging system (Perkin Elmer, Waltham, Mass.) equipped with a 710 to 760 nm excitation filter and 810 to 875 nm emission filter (ICG filter set). Animals were kept in a dark chamber between imaging sessions to minimize bleaching of the fluorescent dye.

The data were analyzed for differences in signal between the target-expressing and control tumors and for selectivity of the ligand. An optimized dose of 10 nmol/kg Dmt-Tic-IR800 was selected for the following studies. For selectivity studies, tumors were established as described above. Mice (n=4) were injected via the tail vein with 10 nmol/kg Dmt-Tic-IR800 in 100 μL, PBS. In vivo fluorescence images were acquired pre-injection and at 24 hours post-injection of the ligand using the Optix-MX3 (Advanced Research Technologies, Inc., a subsidiary of SoftScan Healthcare Group, Montreal, Canada). Animals were positioned on a heating pad and anesthetized using isoflurane (flow 2-2.5 L/min). Fluorescence images were acquired using a scan resolution of 1.5 mm and a 785 nm pulsed laser diode with 40 MHz frequency and 12 ns time window. Animals were kept in a dark chamber between imaging sessions to minimize bleaching of the fluorescent dye.

For lung cancer xenografts with endogenous expression levels, female nu/nu mice 6-8 weeks old were injected s.c. with 20×10⁶ DMS-53 cells in the right flank and 12×10⁶ H1299 cells in the left flank. Tumors were allowed to grow for 3 weeks. Four days prior to imaging the mice were switched to fluorescence imaging feed (AIN 93G). For the dose determination studies, mice were injected with one of three doses of Dmt-Tic-IR800 (160 nmol/kg, 80 nmol/kg, or 40 nmol/kg) in 100 μL, PBS via the tail vein and in vivo fluorescence images were acquired as above using the Xenogen IVIS-200 imaging system. The data were analyzed for differences in signal between the target-expressing and non-expressing tumors and for selectivity of the ligand. An optimized dose of 40 nmol/kg Dmt-Tic-IR800 was selected for the following studies. For selectivity studies, tumors were established as described above. Mice (n=4) were injected via the tail vein with 40 nmol/kg Dmt-Tic-IR800 in 100 μL, PBS and in vivo fluorescence images were acquired as above using the Optix-MX3.

For dose determination assays on the Xenogen IVIS-200, images were analyzed using Living Image Software (v3.2). Image data was analyzed in units of efficiency to enable comparison of the different acquisitions normalized for excitation light levels across the stage. Regions of interest (ROIs) were drawn on the images in the locations of the tumors. Autofluorescence background was determined by measuring the mean tumor fluorescence signal prior to injection. This value was subtracted from the fluorescence signal of the same ROI post-injection to obtain mean values for each ROI on the images. Images acquired on the Optix-MX3 were analyzed using Optix-MX3 Optiview software (v 3.01). Regions of interest (ROIs) were drawn on the images in the locations of the tumors. Autofluorescence background was determined by measuring the mean tumor fluorescence signal prior to injection. This value was subtracted from the fluorescence signal of the same ROI post-injection to obtain mean normalized intensity values for each ROI on the images.

Statistics

Data from the competitive binding assay are represented as mean±SEM. All other data are represented as mean±SD. All statistical analyses were performed with GraphPad Prism v 5.04. Unpaired Student's t test was used to determine the statistical significance of differences between two independent groups of variables. For all tests, p≦0.05 was considered significant.

Radionuclides

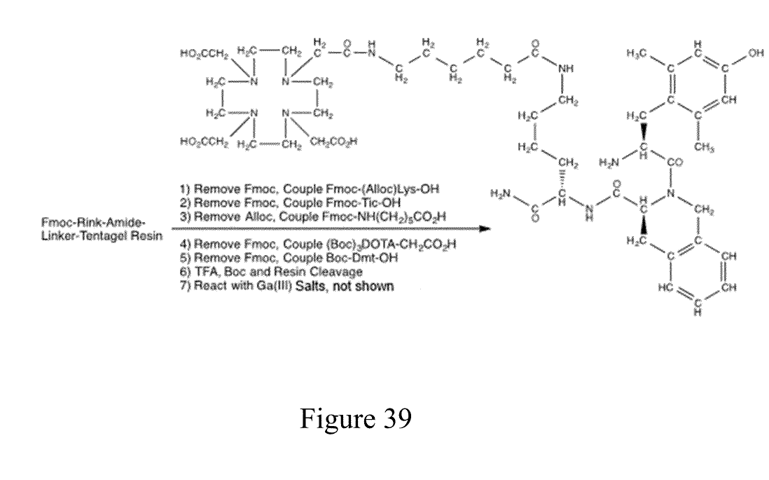

Current molecularly targeted treatments are inhibitors of specific mutations in pathways that promote cancer progression and growth. However, lung cancer rapidly evades these targeted therapies due to the presence of multiple mutations and alternate compensatory signaling pathways. (Gonzalez de Castro D, et al., Clin Pharmacol Ther, 2013, 93(3):252-9; Lackner M R, et al., Future Oncol, 2012, 8(8):999-1014.) By immunohistochemistry (IHC) of patient samples on a tissue microarray assembled by the Moffitt Lung SPORE/Center of Excellence, the inventors have shown that the delta opioid receptor (50R) is overexpressed in 73% of NSCLC and is not expressed in normal lung or other normal tissues of concern for toxicity outside the central nervous system. Hence, an antagonist ligand (Dmt-Tic) that binds with high affinity (0.047 nM Ki) and specificity to the bona-fide lung cancer cell-surface marker, δOR, as a targeting scaffold could carry diagnostic imaging payloads. (Josan J S, et al., Org Lett, 2009, 11(12):2479-82) The cell-surface marker acts as a landing pad for the targeted agent. A positron emission tomography (PET) diagnostic imaging agent made by chelating ⁶⁸Ga into the identical parent compound can be used to non-invasively identify/stratify candidates for therapy and to follow treatment response. Scheme 5 depicts standard Fmoc-based solid-phase-peptide synthesis (SPPS) with Alloc-Lys as an orthogonally protected side chain. (FIG. 39)

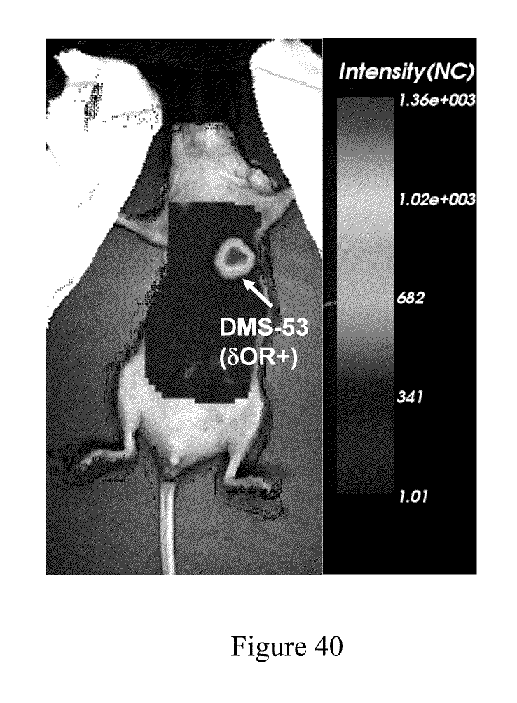

A lead DOTA conjugate of the δOR-specific antagonist ligand Dmt-Tic was developed for imaging of lung cancer. A positron emitting radionuclide (⁶⁸Ga) can be strongly chelated into a DOTA molecule that is conjugated to the Dmt-Tic targeting ligand. The inventors have previously synthesized the fluorescent conjugates, Dmt-Tic-Lys-Cy5 and Dmt-Tic-Lys-IR800, as described above, and have shown that these agents retain high binding affinity, high antagonist activity and selectivity for the δOR in vitro. (Josan J S, et al., Org Lett, 2009, 11(12):2479-82) The inventors have also demonstrated high in vivo tumor selectivity with favorable pharmacokinetic (PK) and biodistribution (BD) profiles, i.e. rapid systemic clearance, does not cross the blood-brain barrier, and has high tumor retention in orthotopic lung tumor models with endogenous expression of the δOR (FIG. 40). Dmt-Tic conjugated to the DOTA chelator (Dmt-Tic-Lys(DOTA)-OH) can be labeled with ^{69/71}Ga as a nonradioactive surrogate for ⁶⁸Ga.

### CONCLUSION

The inventors have synthesized a near-infrared fluorescent agent Dmt-Tic-IR800 and have demonstrated that this agent retains high binding affinity and specificity for the δOR both in vitro and in vivo using a cell line engineered to express the receptor. Lung cancer cell lines have also been identified that endogenously express the δOR and these cell lines can be imaged in a bilateral flank subcutaneous model.

The inventors have evaluated the pharmacokinetics (PK) and biodistribution (BD) of Dmt-Tic-IR800 in endogenous lung cancer cells. The inventors studied Dmt-Tic-IR800 using an orthotopic model of lung cancer since this is a more realistic representation of the clinical problem. The potential for intraoperative guidance by comparing the use of fluorescence-guidance to traditional white-light surgery is also studied. In the clinic, the agent can be used for margin detection during surgery. This capability could improve the rate of complete surgical resection, thereby decreasing the amount of tumor left behind and increasing tumor-free survival.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A molecular probe having affinity for the delta opioid receptor comprising:
a ligand; and
a detection moiety conjugated to the ligand;
wherein the ligand is a synthetic peptide δ-opioid receptor (δOR) antagonist.

2. The molecular probe of claim 1, further comprising a linker molecule wherein the linker molecule is conjugated to both the ligand and the detection moiety to attach the detection moiety to the ligand.

3. The molecular probe of claim 2, wherein the linker molecule is selected from the group consisting of 3-mercaptopropionyl (Mpr), 8-amino-3,6-dioxaoctanyl (Ado), proline-lysine chains, polyethylene glycol oligomers and combinations thereof.

4. The probe of claim 1, wherein the synthetic peptide δ-opioid receptor (δOR) antagonist is Dmt-Tic.

5. The probe of claim 1, wherein the detection moiety is selected from the group consisting of a fluorescent dye and a radionuclide.

6. The probe of claim 5, wherein the detection moiety is a fluorescent dye.

7. The probe of claim 6, wherein the fluorescent dye is selected from the group consisting of Cy5 and IR800CW.

8. A method of detecting lung cancer cells for treatment or removal comprising:
providing a molecular probe directed to a specific marker expressed on the lung cancer cells wherein the markers are selected from the group consisting of CA9, CA12, CTAG2, CXorf61, DSG3, FAT2, KISS1R, GPR87, LYPD3, OPRD1, SLC7A11 and TMPRSS4;
administering the molecular probe to a patient in need thereof; and
imaging the patient with a molecular imaging device capable of detecting a detection signal from the molecular probe;
wherein detection of the detection signal of the molecular probe is indicative of presence of cancer cells.

9. The method of claim 8, wherein the marker is OPRD1.

10. The method of claim 9, wherein the molecular probe is comprised of:
a ligand wherein the ligand is a synthetic peptide δ-opioid receptor (δOR) antagonist;
a linker molecule conjugated to the ligand; and
a detection moiety conjugated to the linker molecule wherein the detection moiety is selected from the group consisting of a fluorescent dye and a radionuclide.

11. The method of claim 10, wherein the synthetic peptide δ-opioid receptor (δOR) antagonist is Dmt-Tic.

12. The method of claim 10, wherein the linker molecule is selected from the group consisting of 3-mercaptopropionyl (Mpr), 8-amino-3,6-dioxaoctanyl (Ado), proline-lysine chains, polyethylene glycol oligomers and combinations thereof.

13. The method of claim 10, wherein the detection moiety is a fluorescent dye.

14. The method of claim 13, wherein the fluorescent dye is selected from the group consisting of Cy5 and IR800CW.

15. A method of detecting lung cancer cells in a patient comprising:
administering a molecular probe to the patient wherein the molecular probe specifically binds to a marker wherein the marker is δ-opioid receptor; and
imaging the patient with a molecular imaging device capable of detecting a detection signal from the molecular probe;
wherein detection of the detection signal of the molecular probe is indicative of presence of cancer cells.

16. The method of claim 10, wherein the molecular probe is comprised of:
a ligand wherein the ligand is a synthetic peptide δ-opioid receptor (δOR) antagonist;
a linker molecule conjugated to the ligand; and
a detection moiety conjugated to the linker molecule wherein the detection moiety is selected from the group consisting of a fluorescent dye and a radionuclide.

17. The method of claim 16, wherein the synthetic peptide δ-opioid receptor (δOR) antagonist is Dmt-Tic.

18. The method of claim 16, wherein the linker molecule is selected from the group consisting of 3-mercaptopropionyl (Mpr), 8-amino-3,6-dioxaoctanyl (Ado), proline-lysine chains, polyethylene glycol oligomers and combinations thereof.

19. The method of claim 16, wherein the detection moiety is a radionuclide.

20. The method of claim 19, wherein the radionuclide is a positron emitting radionuclide.
